(19) United States
(12) Patent Application Publication
(10) Pub. No.: US 2012/0203108 A1
(43) Pub. Date: Aug. 9, 2012

(54) ULTRASONIC DIAGNOSTIC APPARATUS AND IMAGE CONSTRUCTION METHOD

(75) Inventor: Takehiro Tsujita, Tokyo (JP)

(73) Assignee: HITACHI MEDICAL CORPORATION, Tokyo (JP)

(21) Appl. No.: 13/501,025

(22) PCT Filed: Oct. 15, 2010
(86) PCT No.: PCT/JP2010/068135

§ 371 Date: Apr. 9, 2012

(30) Foreign Application Priority Data
Oct. 28, 2009 (JP) 2009-247981

(51) Int. Cl. A61B 8/14; G06K 9/00

(52) U.S. Cl. 600/445; 382/131

(57) ABSTRACT

An ultrasonic diagnostic apparatus is provided with: an ultrasonic probe which is brought into contact with an object to transmit and receive ultrasonic waves; a transmission unit and a reception unit which periodically transmit and receive the ultrasonic waves to and from the object and subject a reflection echo signal from the object to reception processing; a displacement measurement unit which sequentially finds the displacements of a living organism tissue at the position of cross section at which the ultrasonic waves are transmitted to and received from the object; an elasticity image construction unit which sequentially constructs the elasticity images of the living organism tissue; and a three-dimensional image construction unit which constructs a three-dimensional elasticity image.

### TECHNICAL FIELD

The present invention relates to an ultrasonic diagnostic apparatus and an image construction method for generating an elasticity image indicating the hardness or softness of a living organism tissue of an object using ultrasonic waves and constructing a three-dimensional elasticity image on the basis of elasticity images.

### BACKGROUND ART

An ultrasonic diagnostic apparatus is intended to transmit ultrasonic waves to the interior of an object by an ultrasonic probe, receive ultrasonic reflection echo signals corresponding to the structure of a living organism tissue from the interior of the object, and generate an ultrasonic tomographic image such as a B-mode image to display the ultrasonic tomographic image for diagnosis.

It is commonly performed to manually or mechanically press an object with an ultrasonic probe and generate an elasticity image indicating the hardness or softness of a living organism tissue at a cross-sectional plane of the object on the basis of one pair of pieces of RF signal frame data different in measurement time (the amount of pressing).

Patent Literature 1 discloses the process of obtaining a displacement of a living organism tissue caused by a difference in the amount of pressing on the basis of tomographic volume data (multi-slice tomographic image data) obtained by three-dimensional scanning using ultrasonic waves before and after pressing of the object, generating elasticity volume data (multi-slice elasticity image data) indicating the elasticity of the living organism tissue on the basis of the displacement, and generating a three-dimensional elasticity image on the basis of the generated elasticity volume data.

### CITATION LIST

### Patent Literature

### Patent Literature 1

International Publication No. WO 2004/010872

### Patent Literature 2

Japanese Patent Laid-Open No. 2008-259555

### SUMMARY OF INVENTION

### Technical Problem

According to Patent Literature 1, three-dimensional scanning needs to be performed while an ultrasonic probe is fixed in a specific state of pressing, in order to keep the state of pressing against an object before or after the pressing constant. Fixation of the ultrasonic probe for each scanning operation, however, is troublesome. For this reason, multi-slice tomographic images and multi-slice elasticity images are generally measured while pressing force is cyclically changed with an ultrasonic probe by manual operation.

However, if, in a process in which pressing force is cyclically changed, one pair of pieces of RF signal frame data different in measurement time (the pressing force) is measured, and a plurality of elasticity images are sequentially acquired on the basis of pairs of pieces of RF signal frame data, displacement positions of a living organism tissue are different due to a difference in pressing force. Particularly if elasticity volume data is generated on the basis of elasticity images acquired at different positions in a displacement direction of an object and is converted to a three-dimensional image, a difference arises in the displacement direction of the object between elasticity images, and artifacts such as vertical fluctuations in the three-dimensional image are produced. This causes the problem of a reduction in image accuracy.

In order to solve the problem, in Patent Literature 2, tomographic images generated under the same pressing force applied to a living organism tissue are selected from among a plurality of tomographic images, volume data is generated on the basis of elasticity images corresponding to the tomographic images, and a three-dimensional elasticity image is constructed. More specifically, since pressing force is cyclically applied in a displacement direction of an object, there are two images at the same displacement position in the displacement direction, one during push operation and one during pullback operation. For this reason, elasticity volume data is generated by selecting one from among elasticity images obtained during each operation, and a three-dimensional elasticity image is constructed. For example, assume that measurement is performed with three cycles of pressing operation and that pressing force is weaker during pressing operation in the second cycle than in the first and third cycles. In this case, since the displacement state of an image acquired in the second cycle is different from those in the other cycles, an image in a different displacement state may be mixed in a three-dimensional elasticity image, and the three-dimensional elasticity image may suffer from the problem of vertical fluctuations and the like. The same applies to tomographic volume data.

The present invention has a solution to construct a three-dimensional image with reduced artifacts caused by a change in pressing force.

### Problems to be Solved

In order to achieve the above solution, an ultrasonic diagnostic apparatus according to the present invention includes: an ultrasonic probe which transmits/receives ultrasonic waves to/from an object in contact with the object; a transmission/reception unit which performs reception processing on a reflection echo signal from the object and measures RF signal frame data; a displacement measurement unit which obtains a displacement on the basis of the RF signal frame data measured by the transmission/reception unit; an elasticity image construction unit which constructs an elasticity image on the basis of the displacement obtained by the displacement measurement unit; and a three-dimensional image construction unit which generates elasticity image volume data by obtaining a cumulative displacement or a cumulative strain by accumulating the elasticity image constructed by the elasticity image construction unit to generate volume data of the elasticity image and constructs a three-dimensional elasticity image on the basis of the generated volume data.

More specifically, the ultrasonic diagnostic apparatus is adapted to include: the ultrasonic probe which transmits/receives ultrasonic waves to/from the object while being in contact with the object; the transmission/reception unit which, in a process in which pressing force applied to the object by the ultrasonic probe is changed and a cross-sectional position for transmitting/receiving ultrasonic waves to/from the object is moved in a short axis direction, periodically transmits/receives ultrasonic waves to/from the object, performs reception processing on a reflection echo signal from the object, and measures RF signal frame data at the cross-sectional position; the displacement measurement unit which, on the basis of two pieces of RF signal frame data measured by the transmission/reception unit at different measurement times, sequentially obtains a displacement of respective living organism tissue at the cross-sectional position; the elasticity image construction unit which, on the basis of the displacement sequentially obtained by the displacement measurement unit, sequentially constructs an elasticity image of the living organism tissue; and the three-dimensional image construction unit which obtains a cumulative displacement by accumulating a displacement of the living organism tissue in the elasticity image sequentially constructed by the elasticity image construction unit, selects one having the cumulative displacement within a set range from among a plurality of the elasticity images to generate volume data of the selected elasticity image, and constructs a three-dimensional elasticity image on the basis of the generated volume data.

According to the present invention, since volume data of elasticity images having cumulative displacements within a set range is generated, elasticity volume data under substantially uniform pressing force can be generated. That is, since equal cumulative displacements mean that displacement positions in a vertical direction of a living organism tissue or a pressurization/depressurization direction are equal at respective cross-sectional planes, a three-dimensional elasticity image with further reduced artifacts such as vertical fluctuations can be constructed. The term cumulative displacement here refers to a displacement of a living organism tissue from when pressing starts to when a piece of elasticity frame data corresponding to an elasticity image is measured. Assuming that a direction in which the ultrasonic probe is pushed into an object is positive and that a direction in which the ultrasonic probe is pulled back is negative, a cumulative displacement is at the maximum when the ultrasonic probe is pushed into the object to the utmost and is at the minimum when the ultrasonic probe is pulled back to the utmost, i.e., under zero pressure.

A cumulative displacement is obtained from an integrated value of displacements between elasticity frames. Since a displacement of a living organism tissue caused by pressing depends on the hardness of the living organism tissue in the object, parts of the living organism tissue have different displacements. Accordingly, it is desirable to, for example, set a region of interest, obtain an average value of displacements of a living organism tissue in the region of interest, and set the average value as a displacement between elasticity frames. Note that an average value of displacements may be obtained by providing a plurality of sample points in a region of interest, obtaining a displacement between each sample point before pressing and that after pressing, and averaging the displacements. Statistical data such as a median, a variance, or a standard deviation can be used in addition to an average value. Also, displacements obtained by the displacement measurement unit can be assigned to respective pieces of displacement elasticity frame data at corresponding displacements, and the elasticity image construction unit can obtain a cumulative displacement on the basis of the displacements assigned to the pieces of elasticity frame data.

According to the present invention, the state of pressing of an object by the ultrasonic probe need not be fixed. This eliminates the need for a pressure device or the like for fixing the position of the probe and allows manual pressing. The device thus can be constructed with a simple configuration.

As has been described above, use of elasticity images having cumulative displacements within a set range allows construction of a three-dimensional elasticity image with reduced artifacts. However, pieces of elasticity frame data for elasticity images corresponding to a desired cumulative displacement are not always successfully measured at respective slice positions during movement and measurement in the short axis direction. For this reason, the three-dimensional image construction unit is desirably adapted to generate and interpolate an elasticity image corresponding to a desired cumulative displacement on the basis of elasticity images located next in short-axis scan position to the elasticity image corresponding to the desired cumulative displacement and a relationship between cumulative displacements and the short-axis scan positions of the elasticity images, generate volume data including the interpolated elasticity image, and construct a three-dimensional elasticity image on the basis of the generated volume data.

If pressing force is weaker during pressing operation in a second cycle than in first and third cycles, as in the above-described example, there may be no elasticity image corresponding to a desired cumulative displacement in the second cycle. For this reason, the three-dimensional image construction unit can also be adapted to, if an elasticity image corresponding to a desired cumulative displacement is not obtained in one pressing cycle, generate an elasticity image corresponding to the desired cumulative displacement on the basis of elasticity images obtained in respective pressing cycles immediately preceding and following the one pressing cycle and a relationship between cumulative displacements and short-axis scan positions of the elasticity images. Referring to the above example, the elasticity image corresponding to the desired cumulative displacement in the second cycle is generated on the basis of elasticity images corresponding to the desired cumulative displacement in the first and third cycles located nearest in short-axis scan position to the elasticity image corresponding to the desired cumulative displacement. Accordingly, even if there is no elasticity image corresponding to a desired cumulative displacement, a high-accuracy three-dimensional elasticity image with reduced artifacts can be constructed.

The ultrasonic diagnostic apparatus according to the present invention includes a tomographic image construction unit which sequentially constructs tomographic images of the living organism tissue on the basis of a plurality of pieces of RF signal frame data measured by the transmission/reception unit, and the three-dimensional image construction unit associates the plurality of tomographic images output from the tomographic image construction unit with a respective number of the displacements output from the displacement measurement unit, obtains respective cumulative displacements for the tomographic images by accumulating the associated displacements, selects one having the cumulative displacement within a set range from among the plurality of tomographic images to generate volume data of the selected tomographic image, and constructs a three-dimensional tomographic image on the basis of the generated volume data.

With this configuration, a three-dimensional tomographic image with reduced artifacts can be constructed, as in the case of an elasticity image. The three-dimensional image construction unit associates a tomographic image with, for example, a displacement between two tomographic images taken in from the displacement measurement unit. Note that the three-dimensional image construction unit can also be adapted to obtain a displacement from an output from the tomographic image construction unit, instead of a displacement output from the displacement measurement unit. For example, a displacement can be obtained from the number of sample points moved in a pressing direction among one or a plurality of sample points set in a tomographic image.

As in the case of an elasticity image, the three-dimensional image construction unit may be adapted to generate and interpolate a tomographic image corresponding to a desired cumulative displacement on the basis of tomographic images located next in short-axis scan position to the tomographic image corresponding to the desired cumulative displacement and a relationship between cumulative displacements and the short-axis scan positions of the tomographic images and generate volume data including the interpolated tomographic image. Alternatively, the three-dimensional image construction unit may be adapted to, if a tomographic image corresponding to a desired cumulative displacement is not obtained in one pressing cycle, generate and interpolate the tomographic image corresponding to the desired cumulative displacement on the basis of tomographic images obtained in respective pressing cycles immediately preceding and following the one pressing cycle and a relationship between cumulative displacements and short-axis scan positions of the tomographic images. Note that the ultrasonic diagnostic apparatus according to the present invention may be adapted to construct only elasticity images or only tomographic images, perform interpolation processing, and construct a three-dimensional elasticity image or a three-dimensional tomographic image. Both of the elasticity image construction unit and the tomographic image construction unit are not always necessary.

The three-dimensional image construction unit can also be adapted to obtain an elasticity value on the basis of the three-dimensional elasticity image, convert the three-dimensional elasticity image to an image display format (luminance and tone) corresponding to the elasticity value, and superimpose the three-dimensional elasticity image on the three-dimensional tomographic image. With this configuration, a tester can simultaneously observe form-related information and property-related information.

The three-dimensional image construction unit can also be adapted to create a cumulative displacement graph indicating a relationship between the cumulative displacement and a position in a short-axis scan direction of an elasticity image at the cumulative displacement. The three-dimensional image construction unit can further be adapted to include an image display unit which displays a screen including the cumulative displacement graph and at least one of an elasticity image constructed by the elasticity image construction unit and a tomographic image constructed by the tomographic image construction unit, and a three-dimensional elasticity image and a three-dimensional tomographic image constructed by the three-dimensional image construction unit.

With these configurations, a tester can perform measurement while checking each image in real time. If the measurement is not appropriate, the tester can make a correction immediately and need not perform measurement again later. For example, a three-dimensional elasticity image and a three-dimensional tomographic image are sequentially constructed from acquired elasticity images and tomographic images and obtained cumulative displacements, and if the tester checks the three-dimensional images and considers that the accuracy of the images is low, the tester can improve the image accuracy by correcting the manner of manual pressing operation. In this case, it is possible to construct a piece of correction volume data at each frame update and perform real-time display by, for example, defining a reference cumulative displacement.

In this case, the three-dimensional image construction unit may be adapted to display a screen including an elasticity image or a tomographic image at an arbitrary cross-section of the three-dimensional elasticity image or the three-dimensional tomographic image and a composite image of the elasticity image and the tomographic image.

The three-dimensional image construction unit can further be adapted to, when change of pressing force applied to the object and movement of the cross-sectional position in the short-axis scan direction are manually performed while the ultrasonic probe is grasped, extract a displacement caused by imbalance in the pressing force on the basis of the cumulative displacement graph to create a displacement baseline and display the displacement baseline on the cumulative displacement graph displayed on the image display unit.

If a tester gradually decreases or gradually increases pressing force due to unintentional hand movement or the like at the time of measurement, a displacement may include a fixed displacement component without periodicity or a low-frequency displacement component. That is, for example, a displacement decreases with a gradual reduction in pressing force. In a cumulative displacement graph, the change appears in the form of a reduction of a cumulative displacement in the short-axis scan direction at a constant rate. The change can be estimated as a displacement baseline by, e.g., the method of least squares or low order polynomial approximation. The tester can reduce a displacement caused by unintentional hand movement or the like by changing the manner of measurement so as to clear a displacement baseline when the displacement baseline is displayed on a cumulative displacement graph.

In this case, the three-dimensional image construction unit may be adapted to display a warning on the image display unit when a displacement of the displacement baseline exceeds a set value. A tester can correct pressing operation on the basis of the warning.

The three-dimensional image construction unit may also be adapted to construct the three-dimensional elasticity image or the three-dimensional tomographic image on the basis of a cumulative displacement graph obtained by removing the displacement baseline from the cumulative displacement graph. With this configuration, a three-dimensional image can be constructed by removing effects of unintentional hand movement of a tester and the like from cumulative displacements.

The three-dimensional image construction unit can further be adapted to consecutively display three-dimensional elasticity images or three-dimensional tomographic images on the image display unit on the basis of corresponding cumulative displacements. With this configuration, the process of pressing can be observed with a three-dimensional moving image by, for example, consecutively playing back three-dimensional elasticity images or three-dimensional tomographic images in ascending order of cumulative displacement.

A three-dimensional image construction method can be adapted to include: a step of performing reception processing on a reflection echo signal from an object via an ultrasonic probe which transmits/receives ultrasonic waves to/from the object while being in contact with the object and measuring RF signal frame data; a step of obtaining a displacement on the basis of the measured RF signal frame data; a step of constructing an elasticity image on the basis of the obtained displacement; and a step of generating elasticity image volume data by obtaining elasticity image volume data by accumulating the constructed elasticity image, and constructing a three-dimensional elasticity image on the basis of the generated volume data.

More specifically, the three-dimensional image construction method includes: the step of, in a process in which pressing force applied to the object by the ultrasonic probe transmitting/receiving ultrasonic waves to/from the object while being in contact with the object is changed and a cross-sectional position for transmitting/receiving ultrasonic waves to/from the object is moved in a short axis direction, periodically transmitting/receiving ultrasonic waves to/from the object, performing reception processing on a reflection echo signal from the object, and measuring RF signal frame data at the cross-sectional position; the step of, on the basis of two pieces of RF signal frame data measured at different measurement times, sequentially obtaining a displacement of a living organism tissue at the cross-sectional position; the step of, on the basis of the sequentially obtained displacement, sequentially constructing an elasticity image of the living organism tissue; and the step of obtaining a cumulative displacement by accumulating a displacement of the living organism tissue in the sequentially constructed elasticity image, selecting one having the cumulative displacement within a set range from among a plurality of the elasticity images to generate volume data of the selected elasticity image, and constructing a three-dimensional elasticity image on the basis of the generated volume data.

In this case, the three-dimensional image construction method can be adapted to include a step of generating and interpolating an elasticity image corresponding to a desired cumulative displacement on the basis of elasticity images located next in short-axis scan position to the elasticity image corresponding to the desired cumulative displacement and a relationship between cumulative displacements and the short-axis scan positions of the elasticity images and a step of generating volume data including the interpolated elasticity image.

The three-dimensional image construction method can further be adapted to include a step of, if an elasticity image corresponding to a desired cumulative displacement is not obtained in one pressing cycle, generating and interpolating the elasticity image corresponding to the desired cumulative displacement on the basis of elasticity images obtained in respective pressing cycles immediately preceding and following the one pressing cycle and a relationship between cumulative displacements and short-axis scan positions of the elasticity images and a step of generating volume data including the interpolated elasticity image.

The three-dimensional image construction method can also be adapted to include a step of sequentially constructing tomographic images of the living organism tissue on the basis of a plurality of measured pieces of RF signal frame data and a step of associating the plurality of tomographic images with a respective number of the obtained displacements, obtaining respective cumulative displacements for the tomographic images by accumulating the associated displacements, selecting one having the cumulative displacement within a set range from among the plurality of tomographic images to generate volume data of the selected tomographic image, and constructing a three-dimensional tomographic image on the basis of the generated volume data.

The three-dimensional image construction method can also be adapted to include a step of generating and interpolating a tomographic image corresponding to a desired cumulative displacement on the basis of tomographic images located next in short-axis scan position to the tomographic image corresponding to the desired cumulative displacement and a relationship between cumulative displacements and the short-axis scan positions of the tomographic images and a step of generating volume data including the interpolated tomographic image.

The three-dimensional image construction method can further be adapted to include a step of, if a tomographic image corresponding to a desired cumulative displacement is not obtained in one pressing cycle, generating and interpolating the tomographic image corresponding to the desired cumulative displacement on the basis of tomographic images obtained in respective pressing cycles immediately preceding and following the one pressing cycle and a relationship between cumulative displacements and short-axis scan positions of the tomographic images and a step of generating volume data including the interpolated tomographic image.

The three-dimensional image construction method can also be adapted to include a step of obtaining an elasticity value on the basis of the three-dimensional elasticity image, converting the three-dimensional elasticity image to an image display format (luminance and tone) corresponding to the elasticity value, and superimposing the three-dimensional elasticity image on the three-dimensional tomographic image.

The three-dimensional image construction method can also be adapted to include a step of constructing a cumulative displacement graph indicating a relationship between the cumulative displacement and a position in a short-axis scan direction of an elasticity image at the cumulative displacement. The three-dimensional image construction method can further be adapted to include a step of displaying a screen including the constructed cumulative displacement graph and at least one of a constructed elasticity image and tomographic image, and a three-dimensional elasticity image and a three-dimensional tomographic image.

The three-dimensional image construction method can also be adapted to include a step of displaying a screen including an elasticity image or a tomographic image at an arbitrary cross-section of the three-dimensional elasticity image or the three-dimensional tomographic image and a composite image of the elasticity image and the tomographic image.

The three-dimensional image construction method can also be adapted to include a step of, when change of pressing force applied to the object and movement of the cross-sectional position in the short-axis scan direction are manually performed while the ultrasonic probe is grasped, extracting a displacement caused by imbalance in the pressing force on the basis of the cumulative displacement graph to construct a displacement baseline and displaying the displacement baseline on the image display unit.

The three-dimensional image construction method can also be adapted to include a step of displaying a warning on the image display unit when a displacement of the displacement baseline exceeds a set value. The three-dimensional image construction method can also be adapted to include a step of constructing the three-dimensional elasticity image or the three-dimensional tomographic image on the basis of a cumulative displacement graph obtained by removing the displacement baseline from the cumulative displacement graph.

The three-dimensional image construction method can also be adapted to include a step of displaying three-dimensional elasticity images or three-dimensional tomographic images on the image display unit consecutively on the basis of corresponding cumulative displacements.

### Advantageous Effect of Invention

According to the present invention, it is possible to construct a three-dimensional image with reduced artifacts caused by a change in pressing force.

### BRIEF DESCRIPTION OF DRAWINGS

FIG. 1 is a configuration diagram of an ultrasonic diagnostic apparatus according to the present invention.

FIG. 2 is a configuration diagram of a three-dimensional image construction unit.

FIG. 3 is a figure for explaining a relative displacement and a cumulative displacement.

FIG. 4 is a graph showing the relationship between a cumulative displacement and an interpolation frame displacement.

FIG. 5 is a graph for explaining interpolation processing.

FIG. 6 is a graph for explaining interpolation processing when pressing force is weak.

FIG. 7 is an image display example constructed according to the present invention.

FIG. 8 is an image display example constructed according to the present invention.

FIG. 9 is an image display example constructed according to the present invention.

FIG. 10 is an image display example constructed according to the present invention.

FIG. 11 is an image display example constructed according to a conventional method.

FIG. 12 is a schematic view of a three-dimensional image constructed according to the present invention and a three-dimensional image constructed according to a conventional method.

FIG. 13 is a schematic figure of construction of three-dimensional images at respective cumulative displacements.

FIG. 14 is a chart showing the process of processing by the ultrasonic diagnostic apparatus according to the present invention.

FIG. 15 are views for explaining manual pressing operation.

FIG. 16 is an example of the display style for a two-dimensional tomographic image and a cumulative displacement graph.

FIG. 17 is an example of the display style for a two-dimensional tomographic image and a cumulative displacement graph.

FIG. 18 is a graph for explaining interpolation processing in a forward path and a return path.

FIG. 19 is a chart showing the process of processing of a second embodiment of the present invention.

FIG. 20 is an example of the display style for a two-dimensional tomographic image and a cumulative displacement graph.

FIG. 21(a) is a schematic figure of a three-dimensional image and a cumulative displacement graph in the absence of unintentional hand movement of a tester, and FIG. 21(b) is a schematic figure of a three-dimensional image and a cumulative displacement graph in the presence of unintentional hand movement of the tester.

FIG. 22 is a configuration diagram of an ultrasonic diagnostic apparatus according to a third embodiment of the present invention.

FIG. 23 is a figure for explaining a displacement baseline.

FIG. 24 is a schematic view of processing of displacement baseline shifting.

FIG. 25 are graphs for explaining a fourth embodiment of the present invention.

### DESCRIPTION OF EMBODIMENTS

### First Embodiment

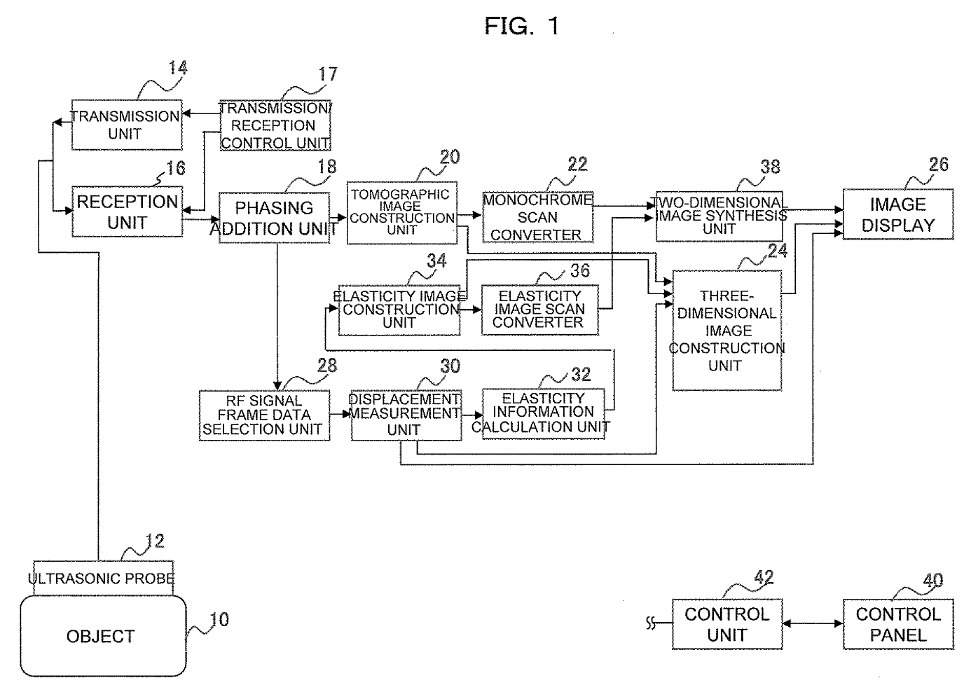

A first embodiment of an ultrasonic diagnostic apparatus will be described below with reference to the drawings. As shown in FIG. 1, an ultrasonic diagnostic apparatus according to the present embodiment includes an ultrasonic probe 12 which transmits/receives ultrasonic waves to/from an object 10 while being in contact with the object 10, a transmission unit 14 which periodically transmits ultrasonic waves to the object 10 in a process in which pressing force applied to the object 10 by the ultrasonic probe 12 is changed and a cross-sectional position for transmitting/receiving ultrasonic waves to/from the object 10 is moved in a short axis direction of the ultrasonic probe 12, a reception unit 16 which performs reception processing on a reflection echo signal from the object 10, a transmission/reception control unit 17 which controls the transmission unit 14 and reception unit 16, and a phasing addition unit 18 which phases and adds pieces of RF signal frame data at cross-sectional positions of a living organism tissue on the basis of reflection echo signals received by the reception unit 16.

The ultrasonic diagnostic apparatus also includes a tomographic image construction unit 20 which constructs a gradation tomographic image (e.g., a monochrome tomographic image) of the object 10 on the basis of RF signal frame data from the phasing addition unit 18 and a monochrome scan converter 22 which converts signals output from the tomographic image construction unit 20 so as to be adapted for display on an image display 26.

The ultrasonic diagnostic apparatus also includes an RF signal frame data selection unit 28 which stores RF signal frame data output from the phasing addition unit 18 and selects at least two pieces of RF signal frame data different in measurement time, a displacement measurement unit 30 which sequentially obtains a displacement of a living organism tissue at a cross-sectional position of the object 10 on the basis of the two pieces of RF signal frame data, an elasticity information calculation unit 32 which obtains elasticity information such as a strain on the basis of the displacement sequentially obtained by the displacement measurement unit 30, an elasticity image construction unit 34 which sequentially constructs a color elasticity image of the living organism tissue from the elasticity information calculated by the elasticity information calculation unit 32, and an elasticity image scan converter 36 which converts signals output from the elasticity image construction unit 34 so as to be adapted for display on the image display 26.

The ultrasonic diagnostic apparatus further includes a two-dimensional image synthesis unit 38 which superimposes a color elasticity image on a monochrome tomographic image, displays the images in parallel, and switches between the images and the image display 26 that displays a composite image obtained by merging the images. The ultrasonic diagnostic apparatus also includes a control panel 40 via which a tester performs various operations and makes various settings and a control unit 42 which controls the respective functional blocks. Note that the control unit 42 is connected to all the functional blocks in FIG. 1 and is capable of outputting control instructions and obtaining pieces of information from the respective functional blocks.

The respective components of the ultrasonic diagnostic apparatus will be described below in detail. The ultrasonic probe 12 is formed such that a plurality of transducers are arranged and has the function of transmitting/receiving ultrasonic waves to the object 10 via the transducers. Movement in the short axis direction of a tomographic image scan plane (in the case of an ultrasonic probe including one-dimensionally arranged oscillating elements, an axis in a direction perpendicular to a direction in which the oscillating elements are arranged) is performed through motor driving that mechanically switches a scan position in the short axis direction of the ultrasonic probe 12 by a motor control unit upon receipt of a control signal or the like from the control unit 42.

If the transducers arranged at a probe head are each cut into k pieces in the short axis direction such that the k pieces are arranged for 1 to k channels, three-dimensional data can also be collected using ultrasonic beams in a long axis direction and the short axis direction along the curvature of the probe head or ultrasonic beams in the long axis and short axis directions generated by electronic focusing. If the ultrasonic probe 12 does not include a mechanism for scanning in the short axis direction, scanning may be performed while the ultrasonic probe 12 is moved freehand in the short axis direction.

The transmission unit 14 generates a wave transmission pulse for driving the ultrasonic probe 12 and causes the ultrasonic probe 12 to generate ultrasonic waves. The transmission unit 14 also has the function of setting a convergent point of transmitted ultrasonic waves to some depth. The reception unit 16 is intended to amplify a reflection echo signal received by the ultrasonic probe 12 with a predetermined gain and generate an RF signal, i.e., a wave reception signal. The phasing addition unit 18 is intended to receive an RF signal amplified by the reception unit 16, subject the RF signal to phase control, form an ultrasonic beam for one or more convergent points, and generate RF signal frame data.

The tomographic image construction unit 20 is intended to receive RF signal frame data from the phasing addition unit 18, subject the RF signal frame data to signal processing such as gain correction, log compression, wave detection, edge enhancement, and filter processing, and obtain tomographic image data. The monochrome scan converter 22 converts the tomographic image data from the tomographic image construction unit 20 to a coordinate system on the image display 26.

The RF signal frame data selection unit 28 stores a plurality of pieces of RF signal frame data from the phasing addition unit 18 and selects one set, i.e., two pieces of RF signal frame data different in measurement time from among the stored group of pieces of RF signal frame data. For example, pieces of RF signal frame data generated in time series, i.e., on the basis of an image frame rate from the phasing addition unit 18 are sequentially stored in the RF signal frame data selection unit 28. The RF signal frame data selection unit 28 selects a stored piece (N) of RF signal frame data as first data and also selects one piece (X) of RF signal frame data from among a previously stored group of pieces (N−1, N−2, . . . , N−M) of RF signal frame data. Note that reference characters N, M, and X each denote an index number assigned to a piece of RF signal frame data, and the index number is assumed to be a natural number.

The displacement measurement unit 30 performs one-dimensional or two-dimensional correlation processing on the selected set, the piece (N) of RF signal frame data and the piece (X) of RF signal frame data and obtains a one-dimensional or two-dimensional displacement distribution regarding a displacement and a motion vector (i.e., the direction and magnitude of the displacement) in a living organism tissue corresponding to each point on a tomographic image.

A block matching method is used here to detect a motion vector. The block matching method includes dividing an image into blocks of, e.g., N×N pixels, focusing on a block in a region of interest, searching for a block most similar to the block of interest in a previous frame, and performing prediction coding, i.e., the process of determining a sample value on the basis of a difference while referring to the blocks.

The elasticity information calculation unit 32 is intended to calculate a strain of a living organism tissue at each point on a tomographic image on the basis of a displacement output from the displacement measurement unit 30 and generate elasticity image frame data based on the strain. At this time, the strain data is calculated by, e.g., spatial differentiation of the displacement of the living organism tissue.

The elasticity image construction unit 34 includes a frame memory and an image processing unit. The elasticity image construction unit 34 stores pieces of elasticity image frame data output in time series from the elasticity information calculation unit 32 in the frame memory and performs image processing on the stored pieces of elasticity image frame data. The elasticity image construction unit 34 also evaluates an error in an elasticity image from pieces of information output from the RE signal frame data selection unit 28, displacement measurement unit 30, or elasticity information calculation unit 32 and applies masking to an image to be output. The elasticity image scan converter 36 converts the coordinates of elasticity image frame data from the elasticity image construction unit 34 to coordinates appropriate for the image display 26.

The three-dimensional image construction unit 24 that is a feature of the present embodiment will now be described. In the present embodiment, the ultrasonic diagnostic apparatus includes the ultrasonic probe 12 that transmits/receives ultrasonic waves to/from the object 10 while being in contact with the object 10, the transmission and reception units 14 and 16 that perform reception processing on a reflection echo signal from the object 10 and measure RF signal frame data, the displacement measurement unit 30 that obtains a displacement on the basis of the RF signal frame data measured by the transmission and reception units 14 and 16, the elasticity image construction unit 34 that constructs an elasticity image on the basis of the displacement obtained by the displacement measurement unit, and the three-dimensional image construction unit 24 that obtains a cumulative displacement or a cumulative strain by accumulating a displacement or a strain in the elasticity image constructed by the elasticity image construction unit 34 to generate elasticity image volume data and constructs a three-dimensional elasticity image on the basis of the generated volume data.

The three-dimensional image construction unit 24 also obtains a cumulative displacement or a cumulative strain by accumulating displacements or strains of a living organism tissue in elasticity images sequentially constructed by the elasticity image construction unit 34 and selects elasticity images having cumulative displacements or cumulative strains within a set range from among the plurality of elasticity images to generate volume data of the selected elasticity images.

The ultrasonic diagnostic apparatus also includes the tomographic image construction unit 20 that sequentially constructs tomographic images of a living organism tissue on the basis of a plurality of pieces of RF signal frame data measured by the transmission and reception units 14 and 16. The three-dimensional image construction unit 24 associates a plurality of tomographic images output from the tomographic image construction unit 20 with displacements output from the displacement measurement unit 30, obtains cumulative displacements of the tomographic images by accumulating the associated displacements, selects tomographic images having cumulative displacements within the set range to generate volume data of the selected tomographic images, and constructs a three-dimensional tomographic image on the basis of the generated volume data.

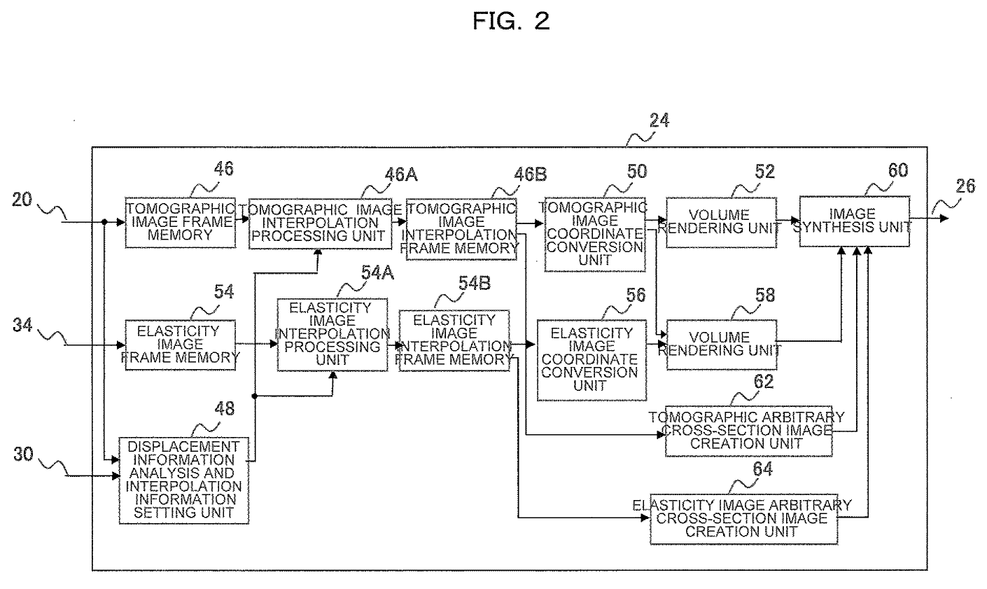

As shown in FIG. 2, the three-dimensional image construction unit 24 includes a tomographic image frame memory 46 which stores tomographic image data output from the tomographic image construction unit 20, a tomographic image interpolation processing unit 46A which performs interpolation processing on the basis of condition setting in a displacement information analysis and interpolation information setting unit 48, a tomographic image interpolation frame memory 46B which stores interpolation frame data created by the tomographic image interpolation processing unit 46A, a tomographic image coordinate conversion unit 50 which performs three-dimensional coordinate conversion on an output from the tomographic image interpolation frame memory 46B, and a volume rendering unit 52 which performs volume rendering of three-dimensional volume data obtained through conversion by the tomographic image coordinate conversion unit 50.

The three-dimensional image construction unit 24 also includes an elasticity image frame memory 54 which stores elasticity image data from the elasticity image construction unit 34, an elasticity image interpolation processing unit 54A which performs interpolation processing on the basis of condition setting in the displacement information analysis and interpolation information setting unit 48, an elasticity image interpolation frame memory 54B which stores interpolation frame data created by the elasticity image interpolation processing unit 54A, an elasticity image coordinate conversion unit 56 which performs coordinate conversion on an output from the elasticity image interpolation frame memory 54B to create three-dimensional volume data, and a volume rendering unit 58 which performs volume rendering of data obtained through coordinate conversion by the elasticity image coordinate conversion unit 56.

An image synthesis unit 60 merges a three-dimensional tomographic image output from the volume rendering unit 52 and a three-dimensional elasticity image output from the volume rendering unit 58 and performs color conversion of an obtained composite image. Note that the tomographic image frame memory 46 and elasticity image frame memory 54 are adapted to store one or more volumes of tomographic image data from the tomographic image construction unit 20 and one or more volumes of elasticity image data from the elasticity image construction unit 34, respectively.

A characteristic function of the displacement information analysis and interpolation information setting unit 48 will now be described. The displacement information analysis and interpolation information setting unit 48 has, as a displacement information analysis function, the function of analyzing an average pressing displacement (average displacement) output from the displacement measurement unit 30 and obtaining a cumulative displacement indicating an absolute displacement position obtained by converting a displacement from a zero-pressure state to a numerical value in terms of a displacement of the object 10 and a relative displacement indicating only a cyclic change in pressing by a tester. Note that the term displacement here refers to a displacement of the object 10 from the zero-pressure state. Although the term displacement is used in the present embodiment, the term can be interchanged with the term strain.

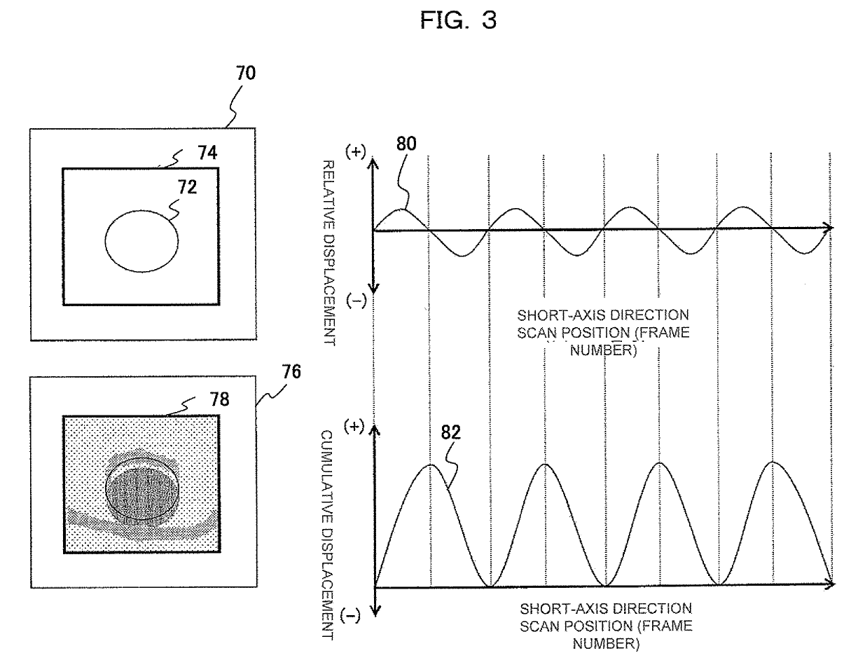

The term average pressing displacement, the term relative pressing displacement, and the term cumulative pressing displacement will now be described with reference to FIG. 3. Note that there is assumed to be no unintentional hand movement at the time of manual pressing operation by a tester. First, the term average pressing displacement will be described. FIG. 3 shows a two-dimensional tomographic image 70 which is obtained by scanning in a displacement direction, a two-dimensional tomographic image 72 showing a hard region present in the two-dimensional tomographic image 70, a region of interest 74 for estimation of elasticity, and a two-dimensional tomographic image 76 when a tomographic image is obtained by pressing the region of interest 74. The term displacement direction in the present embodiment refers to a depth direction.

A region of interest 78 is a tomographic image within a region of interest including a hard region present in the two-dimensional tomographic image 76 and shows displacements at respective pixels between the two-dimensional tomographic image 72 before pressing and the two-dimensional tomographic image 76 after pressing. The pattern of the shaded part indicates that display pixels have different displacements. A displacement of the object 10 caused by pressing depends on the hardness of a living organism tissue in the object 10. Since different displacements are shown for the respective display pixels, the displacements cannot be obtained as an only one value. Accordingly, an average displacement in the region of interest 78 is obtained by adding up displacements in the region of interest 78 and dividing the sum by the number of pixels. In the present embodiment, the average displacement is an average pressing displacement, which will be simply referred to as an average displacement hereinafter.

The term relative pressing displacement will be described next. A relative pressing displacement graph 80 in FIG. 3 has a displacement as the vertical axis and a short-axis direction scan position as the horizontal axis. In the graph 80, a displacement plotted along the vertical axis is an average value of displacements in the region of interest 78. The average value is a positional difference between two consecutive frames and is a deviation of the current frame from the previous frame. Accordingly, the value is a difference between the two frames and is not a pressing displacement obtained as an absolute value. In the present embodiment, a change in average value with respect to the short axis direction is a relative pressing displacement, which will be simply referred to as a relative displacement hereinafter.

Further, the term cumulative pressing displacement will be described. A cumulative pressing displacement graph 82 in FIG. 3 has a cumulative pressing displacement as the vertical axis and a short-axis direction scan position as the horizontal axis. In the graph 82, a displacement plotted along the vertical axis is a cumulative sum of the relative displacements shown in the relative displacement graph 80, which can be rephrased as an integral of a relative displacement. That is, the term cumulative pressing displacement refers to a value obtained by adding up displacements, each of which is obtained between two consecutive frames, from when pressing starts and an absolute displacement from an initial pressing displacement.

As shown in FIG. 3, in a section where a relative displacement is positive, a cumulative pressing displacement increases. The cumulative pressing displacement reaches a positive peak (a convex portion) at a point where the relative displacement is zero. In a section where the relative displacement is negative, the cumulative pressing displacement decreases. The cumulative pressing displacement reaches a negative peak (valley) at a point where the relative displacement is zero. In the present embodiment, a change in displacement with respect to the short axis direction that is obtained by adding up relative displacements from when pressing starts is a cumulative pressing displacement, which will be simply referred to as a cumulative displacement hereinafter.

An interpolation information setting function of the displacement information analysis and interpolation information setting unit 48 will now be described. If manual pressing operation of a tester is a cyclic motion that passes through the same cumulative displacement of the object 10, images having the same cumulative displacement should appear during downward push operation and during upward pullback operation, respectively. However, the period of pressing and the period of ultrasonic scanning actually do not always coincide with each other. In order to obtain an image having a target cumulative displacement, interpolation is performed by multiplying images immediately preceding and following the displacement by respective proportions obtained from the cumulative displacement, and a piece of frame data corresponding to the target cumulative displacement is created. In the present embodiment, the interpolation processing is referred to as target displacement frame interpolation processing.

Movement of a scan position in the short axis direction is performed at times out of sync with the period of pressing and ultrasonic scanning. When an interpolation frame is obtained, a short-axis direction scan position is also interpolated by multiplication by the respective proportions obtained from the cumulative displacement, and a piece of short-axis position frame data corresponding to the target cumulative displacement is also created. In the present embodiment, the interpolation processing is referred to as short-axis position information interpolation processing. The interpolation processing is performed for the entire short-axis scan range, within which ultrasonic scanning is performed. With a plurality of pressing operations, elasticity images and tomographic images at the same cumulative displacement for a plurality of frames can be obtained.

When three-dimensional volume data is created, a set of pieces of two-dimensional frame data, from which the three-dimensional volume data is created, is desirably a set of equally spaced pieces. Elasticity images and tomographic images at the same cumulative displacement for a plurality of frames created by interpolation processing each hold a piece of short-axis direction scan position information. With use of this information, pieces of elasticity frame data and pieces of tomographic frame data at equally spaced short-axis direction scan positions are interpolated by interpolation processing. At this time, an original piece of frame data obtained by manual pressing operation is excluded from an output result. In the present embodiment, the interpolation processing is referred to as equally spaced short-axis frame interpolation processing.

Volume data without vertical fluctuations can be created by performing three-dimensional coordinate conversion processing on a frame data set created by the equally spaced short-axis frame interpolation processing. The process of pressing can be generated as a three-dimensional volume data set by performing the above-described series of processes for each of a plurality of cumulative displacements.

The respective interpolation processes will be described more specifically with reference to FIG. 2. First, the displacement information analysis and interpolation information setting unit 48 sets, as displacement indices, equally spaced cumulative displacements set via the control unit 42 when a tester enters data on the control panel 40. For example, if displacements ranging from +10 μm to −10 μm are to be set in 2 μm increments, {+10 μm, +8 μm, . . . , 0 μm, −2 μm, . . . , −10 μm} is set.

Next, equally spaced short-axis position indices are set on the basis of the number of frames in the short axis direction similarly set from the control panel 40, in order to set equally spaced short-axis direction scan positions. If short-axis direction scanning of the ultrasonic probe is fan-shaped scanning having curvature, the equally spaced short-axis position indices are set in units of angle. For example, if nine frames are desired to be created within a short-axis direction scan range of 10 degree, {+5.00°, +3.75°, . . . , 0.00°, −1.25°, . . . , −5.00°} is set. In the case of parallel scanning, the equally spaced short-axis position indices are set in units of distance. For example, if nine frames are desired to be created within a short-axis direction scan range of 10 mm, {+5.00 mm, +3.75 mm, . . . , 0.00 mm, −1.25 mm, . . . , −5.00 mm} is set. With the above-described processes, the displacement indices and short-axis position indices for interpolation processing relating to cumulative displacements for frames and two-axis interpolation processing relating to short-axis direction scan positions are set.

The target displacement frame interpolation processing will be described. The tomographic image interpolation processing unit 46A and elasticity image interpolation processing unit 54A each creates a piece of frame data using displacement indices set by the displacement information analysis and interpolation information setting unit 48. As for calculation to this end, if the cumulative displacements of frames X0 and X1 are D0 and D1, respectively, and a target displacement Di that is a displacement index satisfies the relation D0<Di<D1, an interpolation frame Xi corresponding to the target displacement Di is generated as Xi=(Di−D0)/(D1−D0)*X1+(D1−Di)/(D1−D0)*X0 by multiplication using factors. Note that although a piece of frame data is denoted here using X, interpolation is actually performed on respective pieces of pixel data on a frame using the same factors.

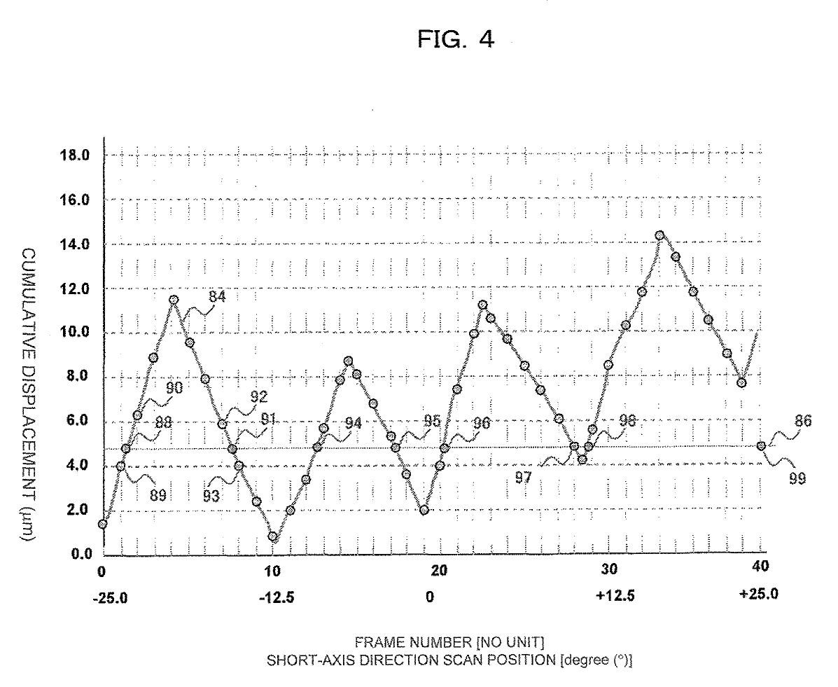

The interpolation processing will be described with reference to FIG. 4. In a cumulative displacement graph 84 in FIG. 4, the vertical axis represents a cumulative displacement, and a point on the graph represents a frame obtained by scanning. The horizontal axis represents a frame number and a short-axis direction scan position expressed as an angle. From pieces of frame data input to the elasticity image interpolation processing unit 54A, frames having a displacement indicated as an interpolation displacement 86 in FIG. 4 are created by interpolation processing using the displacement indices.

The elasticity image interpolation processing unit 54A creates a frame at a position where the cumulative displacement graph 84 and the interpolation displacement 86 cross each other from frames around the frame. For example, a displacement interpolation frame 88 is created using pieces 89 and 90 of frame data, and a displacement interpolation frame 91 is created using pieces 92 and 93 of frame data. Similarly, displacement interpolation frames 94 to 98 are created. If there is no frame beyond the interpolation displacement 86 in the displacement direction like the case of a displacement interpolation frame 99, and there is no displacement interpolation frame on either side of the displacement interpolation frame 99 in the short axis direction, the elasticity image interpolation processing unit 54A inserts an interpolation frame filled with zeros and proceeds to perform subsequent processes. Like the elasticity image interpolation processing unit 54A, the tomographic image interpolation processing unit 46A creates a frame at a position where the cumulative displacement graph 84 and the interpolation displacement 86 cross each other from frames around the frame.

The short-axis position information interpolation processing by the tomographic image interpolation processing unit 46A and elasticity image interpolation processing unit 54A will be described next. As for a short axis position, a piece of short-axis position information corresponding to a frame generated by interpolation using the displacement indices is calculated by interpolation processing. If the short-axis scan positions of the frames X0 and X1 are S0 and S1, respectively, the scan position of the interpolation frame Xi is calculated by interpolation according to the short-axis position indices and is determined by Si=(Di−D0)/(D1−D0)*S1+(D1−Di)/(D1−D0)*S0.

That is, the displacement interpolation frame 88 created by the target displacement frame interpolation processing using the pieces 89 and 90 of frame data in FIG. 4 corresponds to scanning at a position intermediate between the short-axis direction scan position where the piece 89 of frame data is obtained and the short-axis direction scan position where the piece 90 of frame data is obtained. Accordingly, the short-axis direction scan position is also created by interpolation using values similar to the interpolation factors used in the target displacement frame interpolation processing.

The equally spaced short-axis frame interpolation processing by the tomographic image interpolation processing unit 46A and elasticity image interpolation processing unit 54A will be described next. Elasticity images and tomographic images at the same cumulative displacement as a plurality of frames created by interpolation processing each hold a piece of short-axis direction scan position information, as calculated in the short-axis scan position information interpolation processing. A piece of elasticity frame data and a piece of tomographic frame data at a scan position corresponding to the value of a short axis position set by the displacement information analysis and interpolation information setting unit 48 are generated by interpolation processing using the pieces of short-axis direction scan position information.

As for calculation to this end, if the short-axis scan positions of frames Xi0 and Xi1 at the target displacement Di are Si0 and Si1, respectively, and a target short axis position Sij that is a short-axis position index desired to be created by interpolation satisfies the relation Si0<Sij<Si1, an interpolation frame Xij corresponding to the target short axis position Sij is generated as Xij=(Sij−Si0)/(Si1−Si0)*Xi1+(Si1−Sij)/(Si1−Si0)*Xi0 by multiplication using factors.

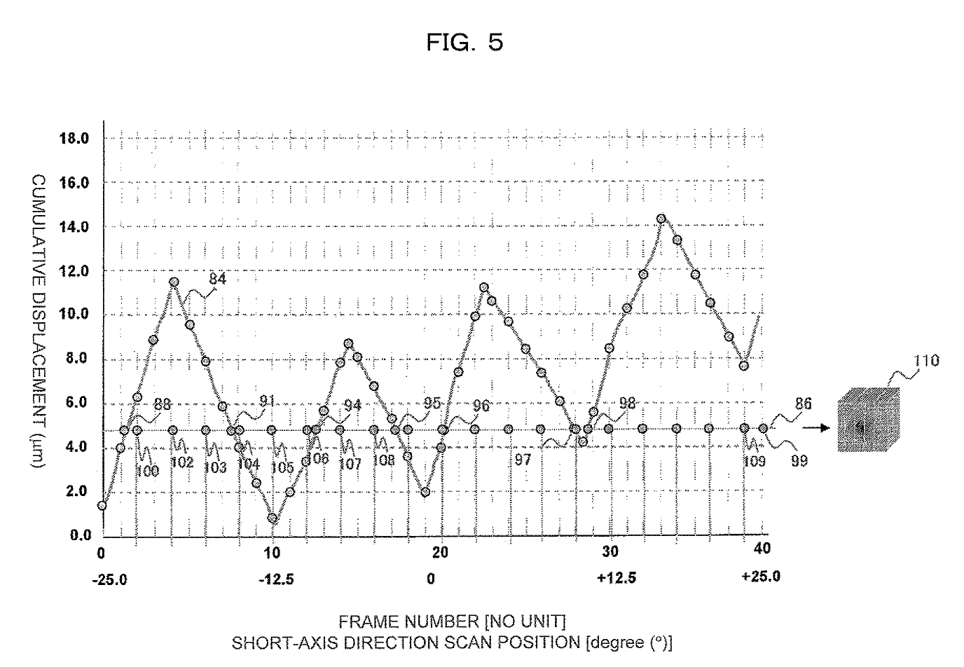

A description will be given with reference to FIG. 5. Equally spaced short-axis direction interpolation frames 100 to 103 are created from the displacement interpolation frames 88 and 91 according to the short-axis position indices, using the displacement interpolation frames 88, 91, and 94 to 98 at the interpolation displacement 86 generated by interpolation using the displacement indices. Equally spaced short-axis direction interpolation frames 104 to 106 are created from the displacement interpolation frames 91 and 94. Similarly, short-axis direction interpolation frames 107 and 108 are created from the displacement interpolation frames 94 and 95, and subsequent short-axis direction interpolation frames up to a short-axis direction interpolation frame 109 are created at even intervals. With this processing, pieces of equally spaced interpolation frame data at the same cumulative displacement required to create a piece 110 of interpolation volume data can be created.

Note that although the target displacement frame interpolation processing and the equally spaced short-axis frame interpolation processing have been described to treat the procedure for creating one interpolation frame as two processes, displacement direction interpolation and short-axis direction interpolation, if there are four pieces of frame data, among which a target displacement index and a short-axis position index are present, it is also possible to combine the above equations and collectively perform the two interpolation processes.

The elasticity information calculation unit 32 described above also has the function of outputting an elasticity image as zero or an invalid value if the elasticity information calculation unit 32 determines that the accuracy of an image which is obtained by calculation on the basis of a displacement from the displacement measurement unit 30 is low (e.g., when manual pressing operation is inappropriate). Accordingly, if data input to the elasticity image interpolation processing unit 54A is a frame including zero or an invalid value, the displacement information analysis and interpolation information setting unit 48 performs interpolation using the immediately preceding and following pieces of frame data and replaces a corresponding piece of elasticity image frame data with a piece of interpolation frame data. Pieces of interpolation frame data can be created without omissions by also replacing an average displacement with a value obtained by the interpolation.

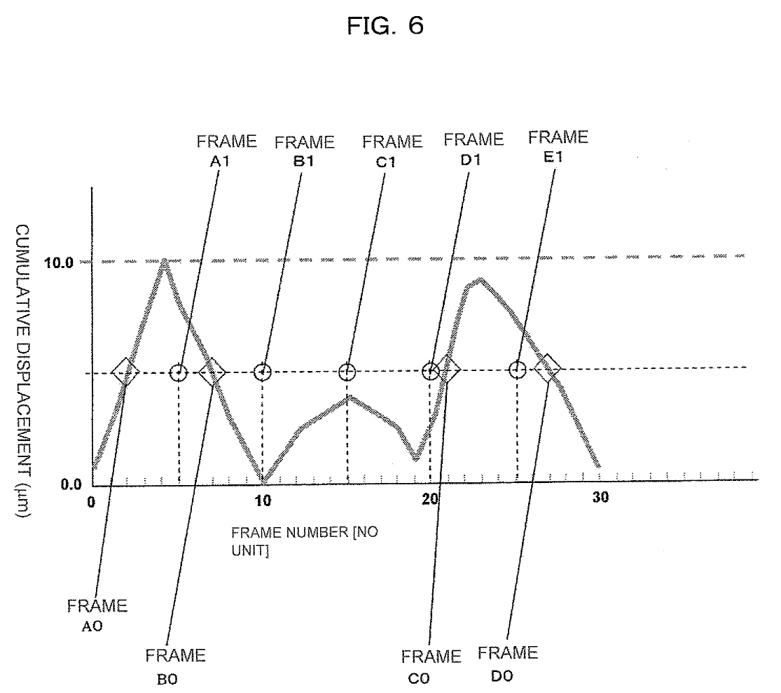

As an example, the equally spaced short-axis frame interpolation processing when data measurement is performed with three cycles of pressing operation with an amplitude of 10 μm, pressing force is weaker during pressing operation in the second cycle than in the first and third cycles, and pressing of only about 3 μm is performed will be described with reference to FIG. 6. If a piece of elasticity volume data at a cumulative displacement of 5 μm is desired to be created, there is no corresponding piece of elasticity frame data in the second cycle. For this reason, pieces A1 to E1 of equally spaced frame data are interpolated using pieces A0 to D0 of frame data created at the same cumulative displacement by the target displacement frame interpolation processing. More specifically, the piece A1 of frame data is interpolated using the pieces A0 and B0 of frame data, the piece E1 of frame data is interpolated using the pieces C0 and D0 of frame data, and the pieces B1, C1, and D1 of frame data are interpolated using the pieces B0 and C0 of frame data.

The tomographic image interpolation processing unit 46A and elasticity image interpolation processing unit 54A store created interpolation frames in the tomographic image interpolation frame memory 46B and elasticity image interpolation frame memory 54B.

The tomographic image coordinate conversion unit 50 and elasticity image coordinate conversion unit 56 convert pieces of interpolation frame data output from the tomographic image interpolation frame memory 46B and elasticity image interpolation frame memory 54B to an orthogonal coordinate system having the mutually orthogonal X-, Y-, and Z-axes to create respective pieces of three-dimensional volume data.

The volume rendering units 52 and 58 each perform volume rendering, maximum or minimum intensity projection, averaging, or the like on volume data present in a view direction of each pixel on a two-dimensional projection plane to be output and create a three-dimensional image from input data.

The volume rendering unit 52 processes tomographic image orthogonal coordinate volume data output from the tomographic image coordinate conversion unit 50 by a publicly known method referred to as so-called volume rendering. The volume rendering unit 52 multiplies each piece of luminance data in the view direction in a three-dimensional tomographic image data set by a luminance-specific transparency value transferred from the control unit 42 and adds the products to create a three-dimensional image that is a mapping on the two-dimensional projection plane. Expressions for a publicly known volume rendering method used in the present embodiment are redefined as follows:

(Expression 1)

Cout=Cout−1+(1−Aout−1)·Ai·Ci (1)

(Expression 2)

Aout=Aout−1+(1−Aout−1)·Ai (2)

In Expression (1), Ci represents the i-th voxel luminance value in the line of sight when a three-dimensional image is viewed from a given point on a two-dimensional projection plane to be created. When N pieces of voxel data lie in the line of sight, a value Cout which is an integrated value of values from when i=0 to when i=N is a final output pixel value. The part Cout−1 represents an integrated value of the 0-th to (i−1)-th value. Also, Ai represents the opacity of the i-th voxel value in the line of sight and takes a value of 0 to 1. The initial values of Cout and Aout are both 0. As given by Expression (2), Aout is cumulatively increased every time a voxel is passed and converges toward 1. Accordingly, if an integrated value Aout−1 of the opacity of the 0-th to (i−1)-th voxels (nearly equal ton) 1, as given by Expression (1), the i-th voxel value Ci is not reflected in an output image.

The relationship between a voxel value and opacity is generally expressed as an opacity table having the horizontal axis representing luminance and the vertical axis representing opacity. Opacity is obtained using a voxel value. As can be seen from the foregoing, in general volume rendering processing, a voxel with high opacity can be taken as a surface, and three-dimensional tomographic image data can be sterically displayed. As a rendering method for transparently visualizing not a surface but an internal structure, Maximum intensity projection that displays only a high-luminance structure in a region of interest, Minimum intensity projection that draws only a low-luminance structure, the method of displaying a cumulative image of voxel values in the view direction (Ray summation), or the like is generally used. The volume rendering unit 52 also has the function of, in the process of rendering processing, selecting whether to enable or disable a piece of voxel data according to a threshold value set from the control panel 40 via the control unit 42.

The volume rendering unit 58 performs volume rendering processing on elasticity image orthogonal coordinate volume data output from the tomographic image coordinate conversion unit 50, like the volume rendering unit 52. In the present embodiment, a change for creating an elasticity value map indicating elasticity values of a surface is made to only ones present at the surface among pieces of voxel data enabled to be displayed at this time.

The volume rendering unit 58 receives tomographic image orthogonal coordinate volume data output from the tomographic image coordinate conversion unit 50 as well as elasticity image orthogonal coordinate volume data output from the elasticity image coordinate conversion unit 56. Since pieces of tomographic image orthogonal coordinate volume data are enabled or disabled according to the threshold value set from the control panel 40, if a piece of tomographic image orthogonal coordinate volume data corresponding to a piece of elasticity image orthogonal coordinate volume data is disabled, the piece of elasticity image orthogonal coordinate volume data is also disabled. That is, the volume rendering unit 58 has the function of performing rendering processing with Expressions (1) and (2) on an input piece of tomographic image orthogonal coordinate volume data only if the piece is enabled by thresholding, thereby creating a three-dimensional image using only pieces of elasticity image orthogonal coordinate volume data at positions corresponding to enabled pieces of tomographic image orthogonal coordinate volume data. In particular, only the surface can be made fully opaque at this time by setting an opacity table set in the volume rendering unit 58 such that the whole range corresponds to opaqueness.

The volume rendering units 52 and 58 may each process data by a publicly known method such as volume rendering, maximum intensity projection, or minimum intensity projection and create a three-dimensional image serving as a mapping on a two-dimensional projection plane, and the image synthesis unit 60 may superimpose one on the other by a publicly known method such as α-blending. Alternatively, three-dimensional images may be created using a method specific to a tomographic image and a method specific to an elasticity image.

A tomographic arbitrary cross-section image creation unit 62 performs the process of cutting an arbitrary cross-section from an interpolation frame set read out from the tomographic image interpolation frame memory 46B. The elasticity image arbitrary cross-section image creation unit 64 performs the process of cutting an arbitrary cross-section from an interpolation frame set read out from the elasticity image interpolation frame memory 54B.

The image synthesis unit 60 merges a three-dimensional tomographic image output from the volume rendering unit 52 and a three-dimensional elasticity image (elasticity value map) output from the volume rendering unit 58. As for a piece of luminance information and a piece of hue information at each pixel of a composite image, each piece of information of the monochrome tomographic image and a corresponding piece of information of the color elasticity image are added in a merging ratio, the result is subjected to RGB conversion, and an image to be displayed on the image display 26 is created. More specifically, a parameter relating to hue (tone) for a corresponding piece of pixel data is determined from the three-dimensional elasticity image, and a parameter relating to luminance for the corresponding piece of pixel data is determined from the three-dimensional tomographic image.

Since a parameter for a blending ratio determined by the control unit 42 determines the adoption ratio between a tomographic image and an elasticity image, the image synthesis unit 60 has the function of determining a parameter relating to chroma from the blending ratio and constructing a three-dimensional image. With this function, the shape of a tomographic image produces an effect such as shades, the coloration of the surface of a three-dimensional image is determined by elasticity values, and a three-dimensional image more accurately indicating a three-dimensional shape and properties than ones generated by general volume rendering can be generated. Alternatively, it is possible to convert only images having arbitrary elasticity values not less than, not more than, or within an elasticity threshold value set from the control panel 40 to a three-dimensional image using the threshold value for both a tomographic image and an elasticity image and display the three-dimensional images.

Note that a rendering method is a technique for creating three-dimensional images from two kinds of pieces of three-dimensional volume data (a tomographic image and an elasticity image) and is a method suitable for the present embodiment intended for an improvement in the accuracy of an image. Such a rendering method, however, is not limited to a system including a data acquisition method according to the present embodiment and relates to a general system intended to construct three-dimensional images for a tomographic image and an elasticity image.

The image synthesis unit 60 also performs superimposition processing such as a-blending of images from the tomographic arbitrary cross-section image creation unit 62 and the elasticity image arbitrary cross-section image creation unit 64 and conversion to a display format and outputs the result to the image display 26 together with three-dimensional images.

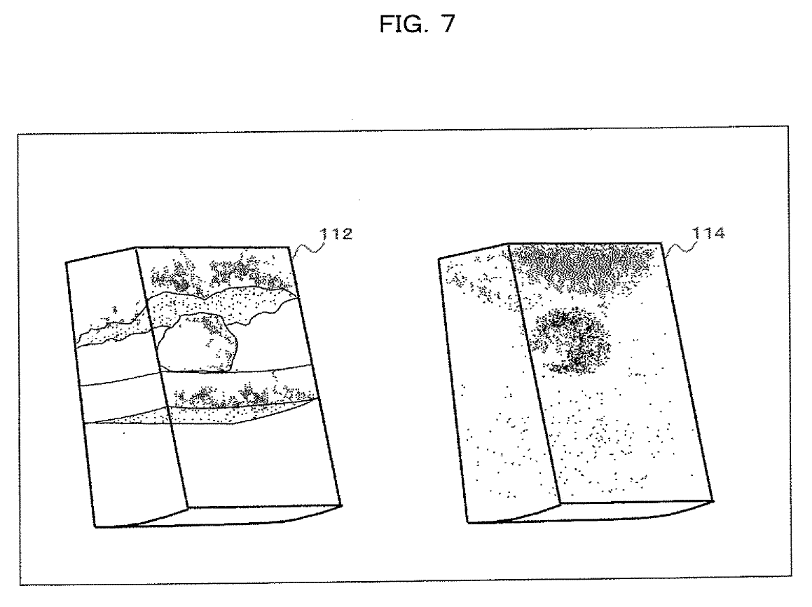

FIG. 7 is an example of an image displayed on the image display 26 and shows a style for simultaneously displaying a three-dimensional tomographic image 112 and a three-dimensional elasticity image 114. The three-dimensional tomographic image 112 is a three-dimensional tomographic image obtained by volume rendering, and the three-dimensional elasticity image 114 is obtained by mapping elasticity values generated by the volume rendering unit 58 onto a three-dimensional tomographic image. Form-related information and property-related information are simultaneously observed by simultaneously displaying a tomographic image and a mapping image as an elasticity image, as shown in FIG. 7.

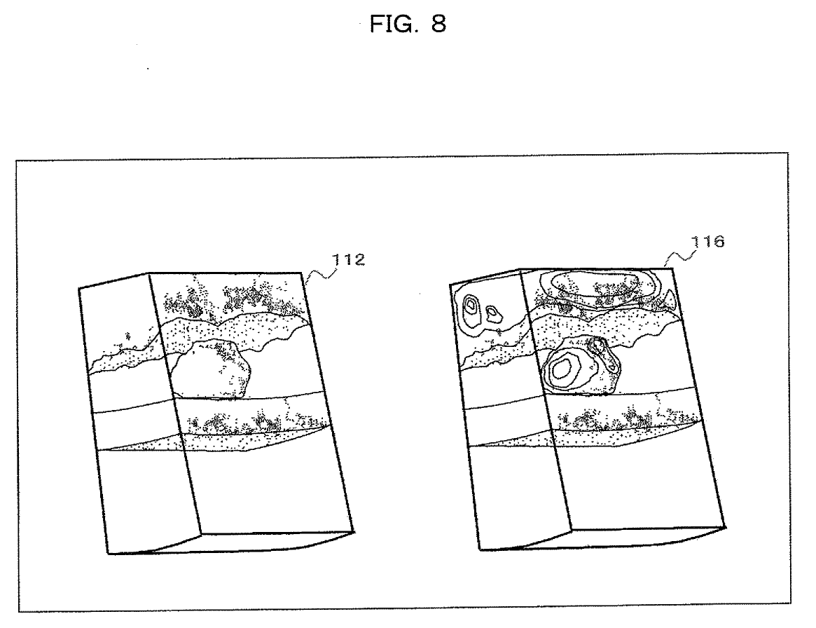

FIG. 8 is another example of the image displayed on the image display 26. A three-dimensional elasticity image 116 is obtained by converting elasticity values to a three-dimensional image by volume rendering. With this figure, form-related information and property-related information are simultaneously observed, as in the display style in FIG. 8.

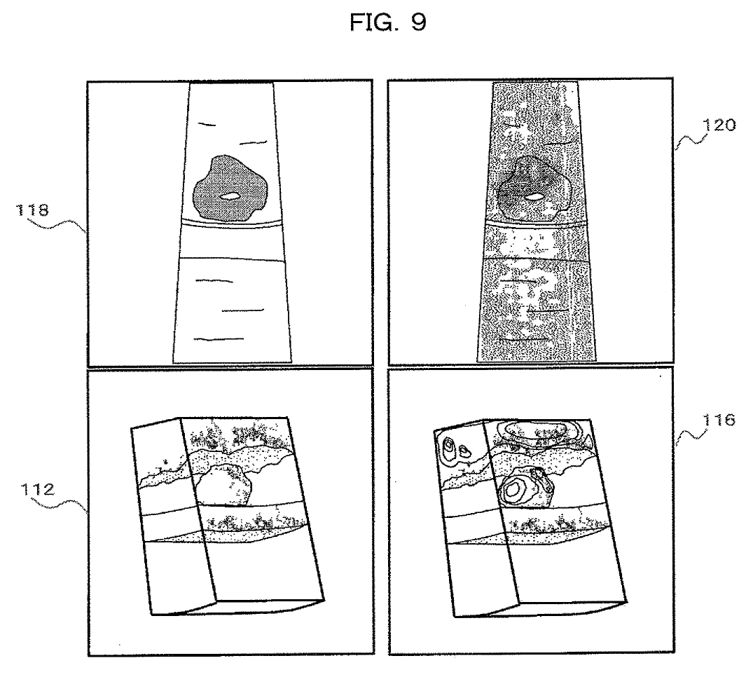

FIG. 9 is still another example of the image displayed on the image display 26 and shows a style for simultaneously displaying a tomographic image arbitrary cross-section image 118 and a composite image 120 of a tomographic image and an elasticity image, in addition to the three-dimensional tomographic image 112 and three-dimensional elasticity image 116. The tomographic image arbitrary cross-section image 118 is a tomographic planar image obtained by cutting tomographic image interpolation volume at the Z-Y plane, and the composite image 120 is obtained by cutting elasticity image interpolation volume at the Z-Y plane and superimposing the result on the tomographic image arbitrary cross-section image 118 by α-blending. A superficial structure and an internal structure and properties are simultaneously observed by simultaneously displaying three-dimensional images for a tomographic image and an elasticity image and arbitrary cross-section images for a tomographic image and an elasticity image, as described above.

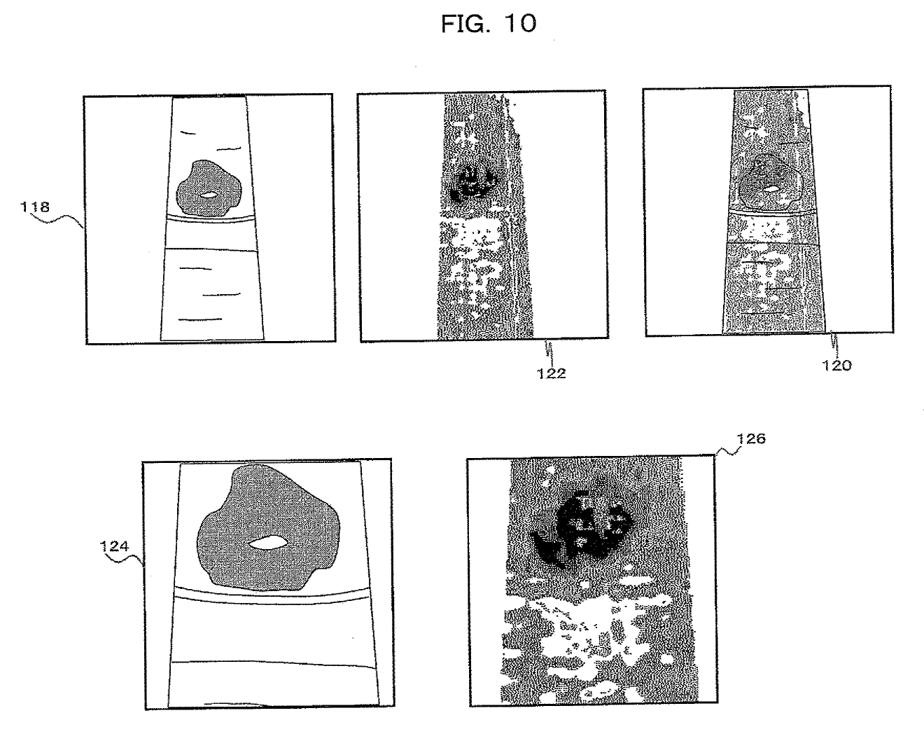

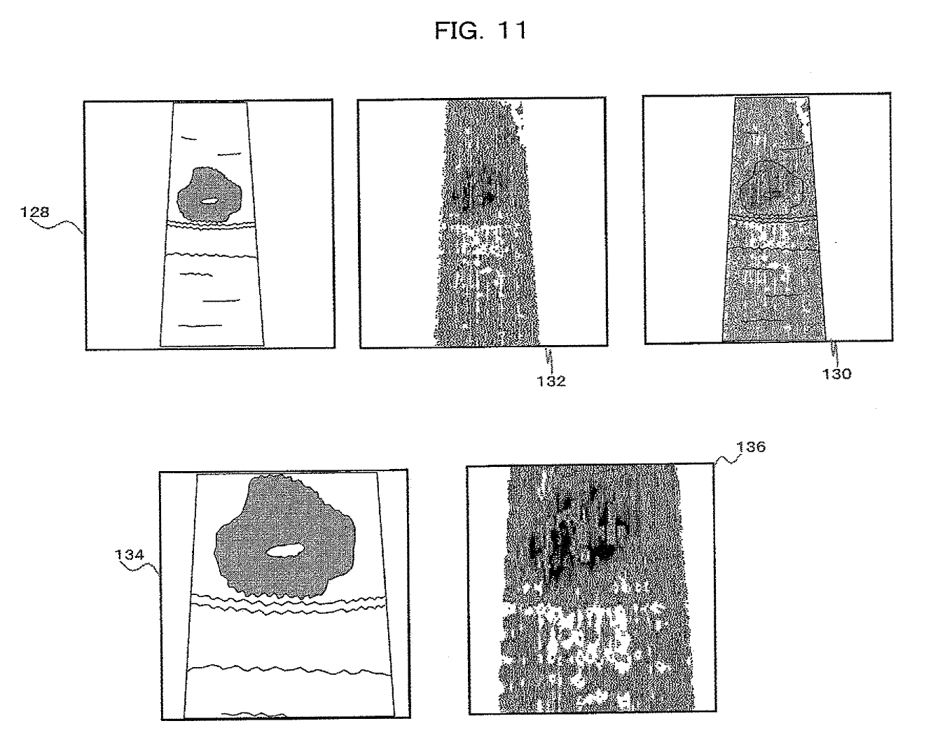

Images constructed according to the present embodiment and images constructed according to a conventional method will be compared with each other with reference to FIGS. 10 and 11. FIG. 10 shows the tomographic image arbitrary cross-section image 118 and an elasticity image arbitrary cross-section image 122 that are each constructed by taking in interpolation volumes characteristic of the present embodiment such that artifacts caused by a change in pressing force appear in the short axis direction of the ultrasonic probe 12, i.e., in a horizontal direction on the image and the composite image 120 that is obtained by superimposing the elasticity image arbitrary cross-section image 122 on the tomographic image arbitrary cross-section image 118 by α-blending. FIG. 10 also shows an enlarged image 124 of the tomographic image arbitrary cross-section image 118 and an enlarged image 126 of the elasticity image arbitrary cross-section image 122.

FIG. 11 shows a tomographic image arbitrary cross-section image 128 and an elasticity image arbitrary cross-section image 132 which are created by a conventional method that takes in all pieces of measured data, a composite image 130 which is obtained by superimposing the elasticity image arbitrary cross-section image 132 on the tomographic image arbitrary cross-section image 128 by α-blending, an enlarged image 134 of the tomographic image arbitrary cross-section image 128, and an enlarged image 136 of the elasticity image arbitrary cross-section image 132.

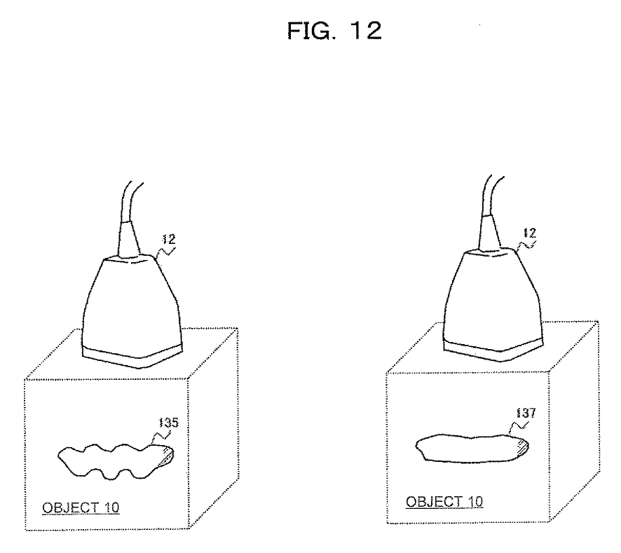

Since pieces of frame data are measured during manual pressing operation that applies vertical pressing in a direction substantially perpendicular to the surface of the body of the object 10, the pieces of frame data include pieces of data measured under different pressures applied to the object 10. If these pieces of data are all taken in to create a three-dimensional image or if ones are unthinkingly selected from among pieces of frame data at respective cross-sectional planes, and the selected pieces of frame data are merged, volume data with vertical fluctuations is created, and the image accuracy is low. It can be seen from FIG. 11 that there are fluctuations on the tomographic image in FIG. 11 and that an image disturbance with streaks is noticeable on the elasticity image. In contrast, it can be seen from FIG. 10 that there are no fluctuations and no image disturbances. FIG. 12 shows a schematic view of a three-dimensional image 135 which is created by a conventional method and a three-dimensional image 137 which is subjected to interpolation processing according to the present embodiment. The three-dimensional image 135 has vertical fluctuations while the three-dimensional image 137 has no fluctuations.

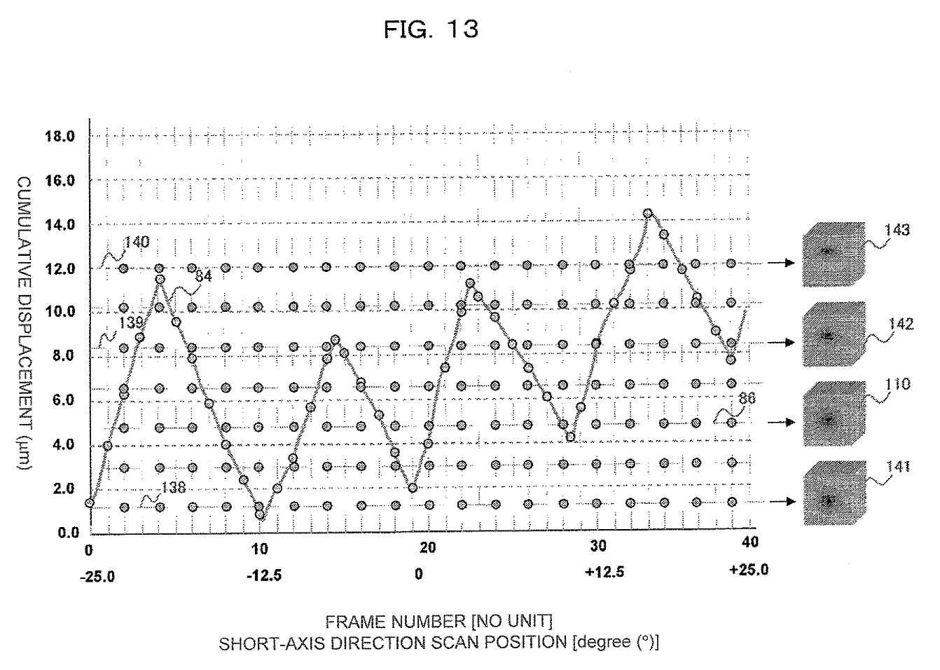

A three-dimensional image and a two-dimensional tomographic image at one cumulative displacement may be displayed at the time of image display. However, the behavior of the object 10 at the time of pressing can be known as if in real time by repeatedly playing back images in the order of the displacement indices created by the displacement information analysis and interpolation information setting unit 48. As shown in FIG. 13, pieces of volume data with different pressing displacements as indicated by pieces 141 and 142 of interpolation volume data, the piece 110 of interpolation volume data, and a piece 143 of interpolation volume data can be created by performing the target displacement frame interpolation processing, the short-axis scan position information interpolation processing, and equally spaced short-axis frame interpolation processing while switching among cumulative displacements 138 and 139, the cumulative displacement 86, and a cumulative displacement 140. The process of pressing can be observed with a three-dimensional moving image by consecutively playing back the pieces of volume data after the end of a test.

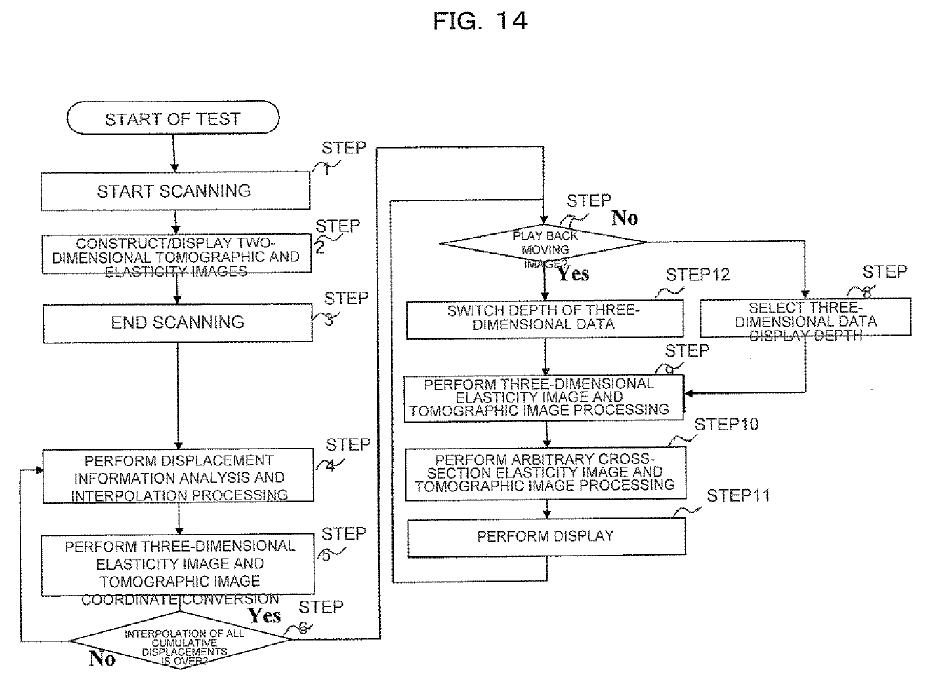

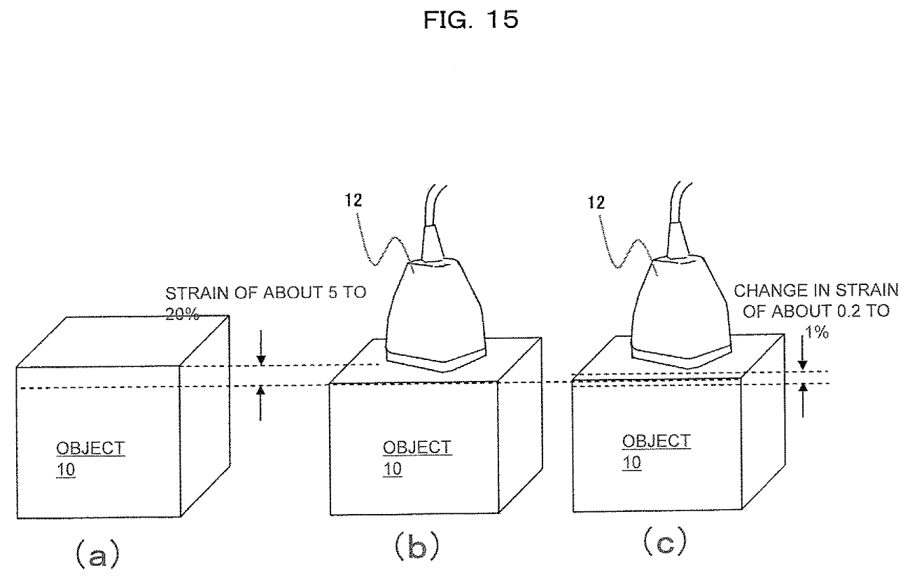

The process of processing by the ultrasonic diagnostic apparatus with the above-described configuration will be described with reference to FIGS. 14 and 15. First, as shown in FIGS. 15(a) to 15(c), a tester brings the ultrasonic probe 12 into contact with the object 10 and vertically and repeatedly operates the ultrasonic probe 12 so as to, for example, cause a strain change of about 0.2% to 1% or 10 μm or less with respect to an initial state with fixed stress applied so as to cause a strain of about 5% to 20% (step 1).

During the operation, ultrasonic waves are transmitted/received, and a two-dimensional tomographic image and a two-dimensional elasticity image are constructed and displayed on the image display 26. Display of a two-dimensional composite image of a tomographic image and an elasticity image is performed in real time for each frame, and the tester can recognize the success or failure of manual operation during pressurization operation. If an elasticity image has not been successfully obtained, the tester can interrupt operation and retry operation (step 2). When scanning of a preset scan range is over, the tester stops transmission/reception of ultrasonic signals (step 3).

If the tester determines that manual pressing operation is appropriately performed while checking the image display 26, the tester inputs a start signal from the control panel 40, starts movement in the short-axis direction, and starts collection of pieces of three-dimensional frame data. Next, the three-dimensional image construction unit 24 performs displacement information analysis and interpolation processing (step 4) and three-dimensional elasticity image and tomographic image coordinate conversion (step 5), and pieces of three-dimensional volume data are constructed. The series of processes is performed for each displacement. When the interpolation processing for all cumulative displacements is over to have pieces of three-dimensional volume data at each displacement, overall interpolation processing ends (step 6).

Next, the tester selects moving image playback or still image playback (step 7). If still image playback is selected, a pressing displacement for a three-dimensional image which is desired to be displayed is automatically or manually set (step 8), one or both of three-dimensional elasticity image and tomographic image processing (step 9) and arbitrary cross-section elasticity image and tomographic image processing (step 10) are performed, and the result is displayed on the image display 26 (step 11).

If the tester selects moving image playback, the current displacement for the created pieces of three-dimensional volume data at the pressing displacements is switched in order (step 12), one or both of the three-dimensional elasticity image and tomographic image processing (step 9) and the arbitrary cross-section elasticity image and tomographic image processing (step 10) are performed, as in the case of a still image, and the result is displayed on the image display 26 (step 11). If stoppage of moving image playback is not selected during the playback, three-dimensional images in the process of manual pressing operation can be observed as if the images were real-time images while the current displacement for three-dimensional volume data is switched in order (step 12).

As has been described above, according to the present embodiment, only elasticity images having cumulative displacements equal to a set value can be selected, and volume data can be generated from the elasticity images. That is, since equal cumulative displacements mean that displacement positions in a vertical direction of a living organism tissue or a pressurization/depressurization direction are equal at respective cross-sectional planes, a three-dimensional elasticity image with further reduced artifacts such as vertical fluctuations can be constructed. Also, according to the present embodiment, the state of pressing against the object 10 by the ultrasonic probe 12 need not be fixed. This eliminates the need for a pressure device or the like for fixing the position of the ultrasonic probe 12 and allows manual pressing. The device thus can be constructed with a simple configuration.

Even if a piece of elasticity frame data for an elasticity image corresponding to a desired cumulative displacement is not measured at respective slice positions during movement and measurement in the short axis direction or even if there is no elasticity image corresponding to the desired cumulative displacement in a cycle with weak pressing force, a high-accuracy three-dimensional elasticity image with reduced artifacts can be constructed using a piece of elasticity volume data generated by interpolation processing.

Additionally, according to the present embodiment, three-dimensional tomographic image with reduced artifacts can be constructed, as in the case of an elasticity image. The three-dimensional image construction unit 24 can also be adapted to obtain a displacement from an output from the tomographic image construction unit 20, instead of a displacement output from the displacement measurement unit 30. Note that only elasticity images or tomographic images may be constructed and that interpolation and construction of a three-dimensional image may be performed using the images. Also, note that both of the elasticity image construction unit 34 and the tomographic image construction unit 20 are not always necessary.

Since the three-dimensional image construction unit 24 is adapted to obtain elasticity values on the basis of a three-dimensional elasticity image and superimpose the three-dimensional elasticity image in an image display format (luminance and tone) corresponding to the elasticity values on a three-dimensional tomographic image, a tester can simultaneously observe form-related information and property-related information.

Since the three-dimensional image construction unit 24 is also adapted to consecutively display three-dimensional elasticity images on the image display unit on the basis of corresponding cumulative displacements, the process of pressing can be observed with a three-dimensional moving image by consecutively playing back three-dimensional elasticity images in ascending order of cumulative displacement.

The present embodiment has been described above. The present invention, however, is not limited to this, and the configuration of the present embodiment can be appropriately changed and used. For example, it is also possible to further provide a pressure measurement unit, calculate an elastic modulus corresponding to each point on a tomographic image from a displacement output from the displacement measurement unit 30 and a measured pressure value, and generate elasticity image frame data on the basis of the elastic moduli.

Elastic modulus data is calculated by dividing a change in pressure by a change in strain. For example, letting L(X) be a displacement measured by the displacement measurement unit 30 and P(X) be a measured pressure, a strain ΔS(X) can be calculated by spatial differentiation of L(X) and thus can be obtained using the equation ΔS(X)=ΔL(X)/ΔX. A Young's modulus Ym(X) of elastic modulus data is calculated by the equation Ym=ΔP(X)/ΔS(X). Since the elastic modulus of a living organism tissue corresponding to each point of a tomographic image is obtained from the Young's modulus Ym, pieces of two-dimensional elasticity image data can be consecutively obtained. Note that Young's modulus is the ratio of a simple tensile stress applied to an object to a strain occurring parallel to the tension.

An average displacement may be obtained by providing a plurality of sample points in a region of interest, obtaining a displacement between each sample point before pressing and that after pressing, and averaging the displacements. Note that statistical data such as a median, a variance, or a standard deviation can be used in addition to an average value.

It is also possible to perform interpolation processing of a tomographic image, generate tomographic volume data, and construct a three-dimensional tomographic image, in the same manner as in the case of an elasticity image. As for a displacement between tomographic images in this case, for example, a displacement estimated from a distance when the correlation value between frames is at its peak detected using correlation processing of the tomographic images or a displacement estimated from a movement distance as a change in measured image barycenter in a pressing direction can be used as an alternative to a displacement calculated by the displacement measurement unit 30.

The present embodiment has further described a method for estimating a pressing displacement from a displacement and creating volume data without positional shifts. If a deviation of a pressing displacement occurs due to an excessive press or an excessive pull in the process of pressing, even interpolation does not allow creation of accurate three-dimensional volume data. In order to prevent this, a cumulative displacement in the perpendicular direction caused by pressing can be displayed in real time.

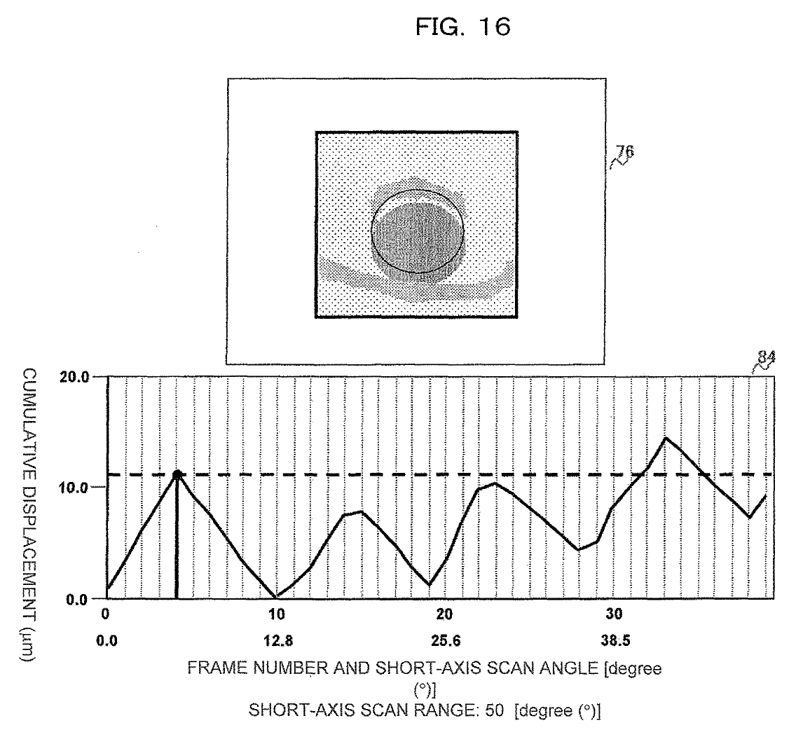

A cumulative displacement can be easily expressed as a cumulative value obtained by adding up values of a time-varying total displacement in the perpendicular direction caused by manual pressing operation. FIG. 16 is a representation of the two-dimensional tomographic image 76 and the cumulative displacement graph 84 in one screen. In interpolation processing according to the present embodiment, pressing is most preferably performed so as to always have the same amplitude from a first data capture range. Accordingly, a displacement when the operation shifts from first push operation to pullback operation is set as a reference, and a straight line horizontal to a direction of time is displayed so as to cross the total displacement. With the graph, a tester can measure good frame data by performing pressing such that a cumulative displacement moves between the zero line and above the total displacement at the time of initial pressing. This allows an increase in test efficiency and an improvement in image quality.

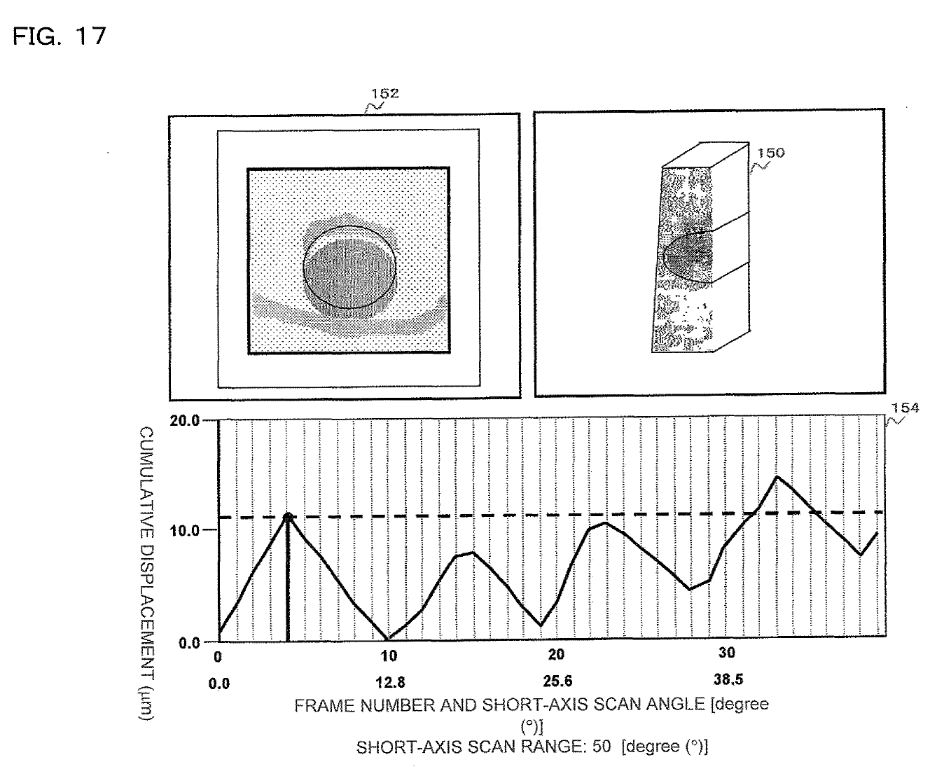

FIG. 17 is a screen on which a three-dimensional image 150 with a short-axis cross-section facing front that is obtained without correction processing according to the present embodiment during manual pressing operation or a cross-section image at a short-axis cross-section cut from three-dimensional data, a general two-dimensional image 152, and a cumulative displacement graph 154 are simultaneously displayed. According to this display style, not only a displacement can be observed from the graph, but also a three-dimensional image being reconstructed can be observed. Since a tester can recognize from an image that pressing is insufficient, the tester can stop scanning without waiting for the end of set scanning and start scanning again. The three-dimensional image 150 or a cross-section image at an arbitrary cross-section at this time is not limited to a short axis plane. A rotation angle can be arbitrarily changed, and a three-dimensional image or a cross-section image at an arbitrary angle can also be displayed.

In the present embodiment, in order to reduce a deviation of a region of interest caused by unintentional hand movement of a tester, it is desirable to move the ultrasonic probe 12 for scanning in the short axis direction only once and create, by interpolation processing using a plurality of obtained frames, three-dimensional data free of a deviation of a displacement caused by pressing. However, if unintentional hand movement can be suppressed, the accuracy can be further improved by using results of a plurality of scans. In this case, respective pieces of frame data at a plurality of planes including the same location can be obtained by performing scanning in two forward and reverse directions or in one direction a plurality of times as operation in the short axis direction. Such pieces of frame data are obtained at the same short-axis direction scan positions, regardless of the number of times of scanning.

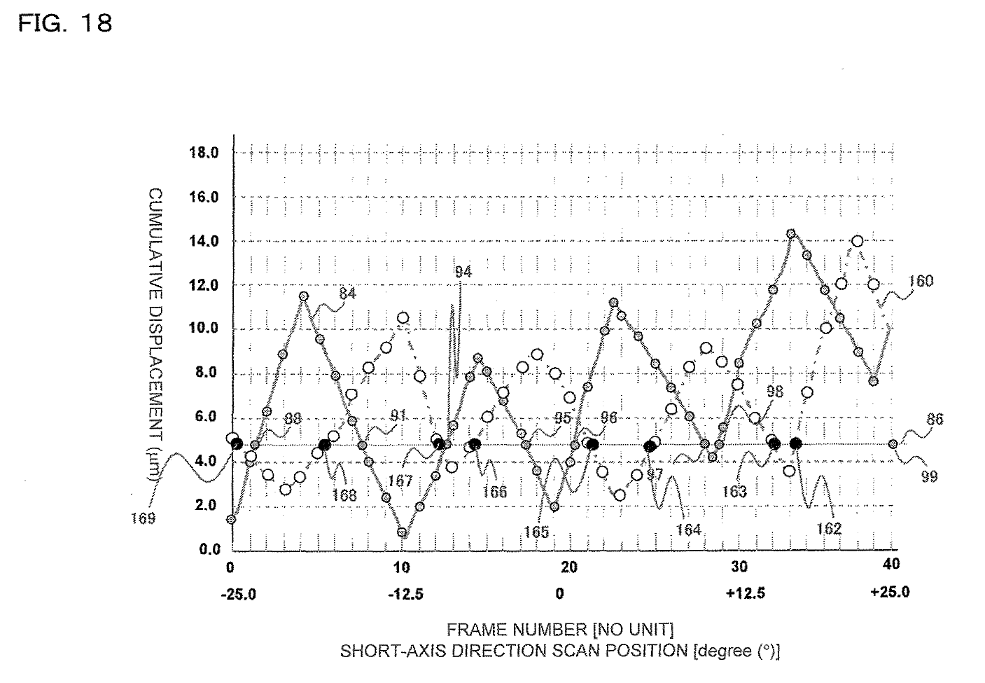

FIG. 18 shows a graph having the horizontal axis representing a short-axis direction scan position when two forward and reverse operations are performed in the short axis direction and the vertical axis representing a cumulative displacement. In FIG. 18, the cumulative displacement graph 84 indicated by a solid line and gray circles is for a forward path while a cumulative displacement graph 160 indicated by an alternate long and short dash line and white circles is for a return path. When interpolation frames are to be created along the interpolation displacement 86 using pieces of scan data in the return path, interpolation frames are created along black circles 162 to 169 on the interpolation displacement line 86 by return scanning, in addition to interpolation by forward scanning. Since interpolation frames are created more densely, a high-accuracy three-dimensional image can be created.

In particular, after the interpolation frame 98 in the forward path, interpolation frames cannot be created, the interpolation frame 99 filled with zeros is present, and interpolation frames which approach zero toward the interpolation frame 99 are only created between the interpolation frame 98 and the interpolation frame 99 by the equally spaced short-axis frame interpolation processing. A three-dimensional image with actual data over a wider scan range can be created by performing the equally spaced short-axis frame interpolation processing using the interpolation frames 162 and 163.

### Second Embodiment

A second embodiment will now be described. In the first embodiment, after all pieces of frame data are measured, the interpolation processes are started. In contrast, in the present embodiment, interpolation volume data is created and displayed in real time. Accordingly, referring to FIG. 2, a tomographic image interpolation processing unit 46A, an elasticity image interpolation processing unit 54A, and a displacement information analysis and interpolation information setting unit 48 are different in operation from those in the first embodiment.

In the present embodiment, a piece of interpolation volume data at one displacement is created to allow real-time display. The number of pressing displacements to set by the displacement information analysis and interpolation information setting unit 48 is thus only one. A pressing displacement to be displayed may be manually set via a control panel 40 before the start of ultrasonic scanning or a frame where a pressing direction is first reversed may be detected from displacements, and ½ the cumulative displacement of the frame may be automatically set as the display pressing displacement, after the start of ultrasonic scanning.

After the pressing displacement is determined, pressing operation is continued during movement in a short axis direction. Displacements are displayed while the pressing displacement is skipped. When frames immediately preceding and following the pressing displacement are both generated, the interpolation factors described above are instantly determined from the cumulative displacements of the immediately preceding and following frames and displacement indices, interpolation frames at one of the displacement indices equal to the pressing displacement are generated, and short axis positions are also recalculated by interpolation.

When scanning of the whole of a short-axis scan range is over, i.e., when all interpolation frames are created, pieces of interpolation volume data are created according to short-axis position indices set by the displacement information analysis and interpolation information setting unit 48. A tomographic image coordinate conversion unit 50 and an elasticity image coordinate conversion unit 56 convert pieces of interpolation volume data output from a tomographic image interpolation frame memory 46B and an elasticity image interpolation frame memory 54B from pieces of scan line data to data in an orthogonal coordinate system having the mutually orthogonal X-, Y-, and Z-axes. Like the first embodiment, three-dimensional images are created from the created pieces of interpolation volume data by volume rendering and are output to an image display 26. By continuing the series of processes during scanning, a three-dimensional tomographic image and a three-dimensional elasticity image at a given pressing displacement manually or automatically set can be displayed in real-time during scanning.

In the present embodiment as well, images as shown in FIG. 17 can be displayed. Not only that, but the present embodiment is capable of interpolation calculation before the end of scanning of one volume. Accordingly, although a three-dimensional image without correction processing is displayed in FIG. 17, the present embodiment can create three-dimensional volume data at a set pressing displacement by correction processing at any time and observe a three-dimensional image being reconstructed or an arbitrary cross-section cut from the three-dimensional volume data.

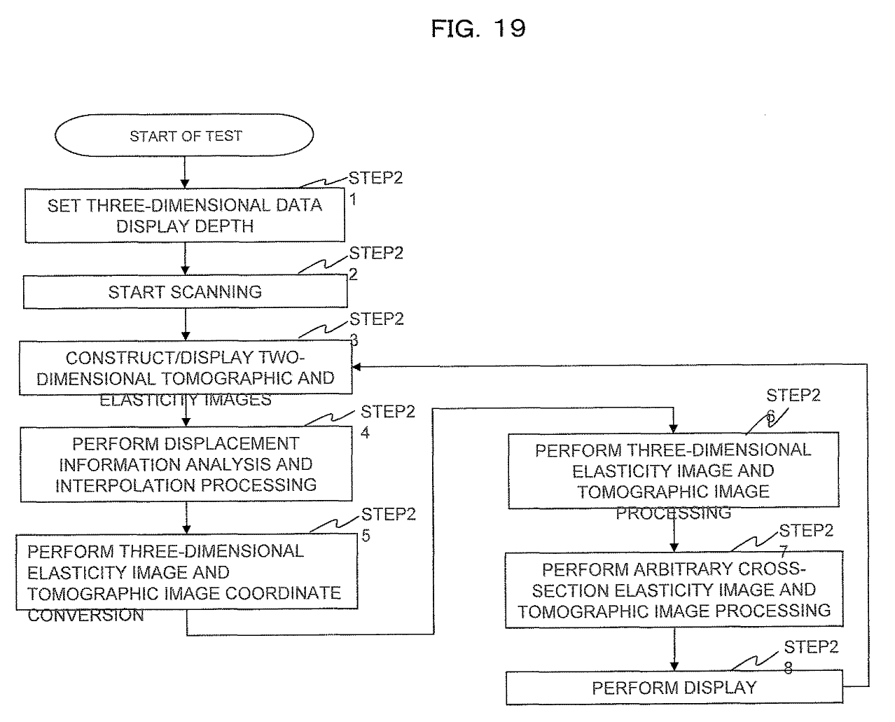

The flow of operation and processing in the present embodiment will be described next with reference to FIG. 19. A tester sets a cumulative displacement to be three-dimensionally represented or makes settings for automatically detecting the cumulative displacement in advance (step 21). When the tester starts scanning (step 22), an interpolation frame at the cumulative displacement set in advance is created by interpolation processing (step 24) while a two-dimensional tomographic image and a two-dimensional elasticity image are constructed and displayed on a screen (step 23). Three-dimensional elasticity image and three-dimensional tomographic image coordinate conversion is performed (step 25), and pieces of three-dimensional volume data are constructed.

The pieces of three-dimensional volume data are input to one or both of three-dimensional elasticity image and tomographic image processing (step 26) and arbitrary cross-section elasticity image and tomographic image processing (step 27) at any time, and images are displayed on the image display 26 (step 28). Since real-time processing is performed in the second embodiment, creation and display of three-dimensional images are continued until the tester ends the test.

As has been described above, according to the present embodiment, a tester can perform measurement while checking images, a cumulative displacement graph 84, an elasticity image, a tomographic image, a three-dimensional elasticity image, and a three-dimensional tomographic image in real time. If the measurement is not appropriate, the tester can make a correction immediately and need not perform measurement again later.

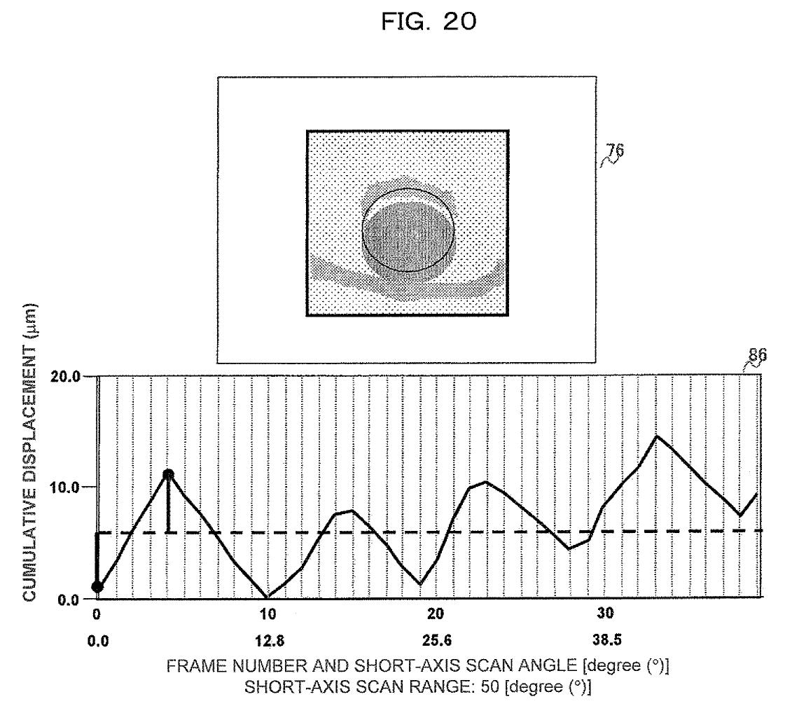

The present embodiment has been described above. The process of displaying a cumulative displacement in a perpendicular direction caused by pressing operation in real time, like the first embodiment, is conceivable. In interpolation processing according to the present embodiment, it is most preferable that a graph consistently pass through the same cumulative displacement. Accordingly, as shown in FIG. 20, ½ a displacement when pressing starts, and the operation shifts from first push operation to pullback operation, i.e., an intermediate displacement in pressing scanning is set as a reference, and a straight line horizontal to a direction of time is displayed so as to cross the displacement. With the graph, a tester can obtain good interpolation volume data by performing pressing such that the graph passes through the line at the intermediate displacement. This allows an increase in test efficiency and an improvement in image quality.

Note that the first embodiment describes interpolation processing after the end of scanning. If a displacement to be three-dimensionally represented is fixed to a given displacement, a piece of three-dimensional volume data at the set cumulative displacement can be created by correction processing at any time, and a three-dimensional image being reconstructed or an arbitrary cross-section cut from three-dimensional data can be observed in real time, like the present embodiment. The tester can know the process of pressing from a three-dimensional image or an arbitrary cross-section image.

### Third Embodiment

A third embodiment will be described next. In the first and second embodiments, frame data is desirably measured with a constant amplitude and a constant period during several cycles in the process of pressing. However, measurement is not always performed in a constant state. A displacement caused by something other than pressing, such as a line composed of an aperiodic or low-frequency displacement component caused when a tester presses with gradually increasing force or gradually decreasing force by unintentional hand movement, may be included. The line is referred to as a displacement baseline in the present embodiment.

A displacement baseline can be estimated from cumulative displacements at all obtained frames by the method of least squares or low order polynomial approximation in a displacement information analysis and interpolation information setting unit 48. As another method for estimating a displacement baseline waveform, a Fourier transform is performed on the waveform of a change in relative displacement with respect to a short axis direction or the autocorrelation waveform of the waveform of a change in relative displacement with respect to the short axis direction, and frequency components of manual pressing operation are specified by detecting the maximum spectrum on the frequency axis. Next, a Fourier transform is performed on the waveform of a change in cumulative displacement with respect to the short axis direction, the estimated frequency components of manual pressing operation are removed from the frequency axis, and conversion to time signals is performed by an inverse Fourier transform. The displacement baseline waveform can also be estimated by the series of processes.

The displacement information analysis and interpolation information setting unit 48 has the function of warning a tester if a variation in the estimated displacement baseline waveform or the magnitude of the spectrum exceeds a set value and can urge the tester to reacquisition. The set value may be set in advance or a value obtained by multiplying the magnitude of a cumulative displacement caused by manual pressing operation that is obtained by removing the displacement baseline waveform from the cumulative displacement by a fixed proportion may be used as a reference.

The displacement information analysis and interpolation information setting unit 48 obtains a result of warning the tester in the above-described manner. The displacement information analysis and interpolation information setting unit 48 can also perform correction processing using, as cumulative displacements, pressing displacements obtained by removing the displacement baseline waveform from cumulative displacements, if the tester desires to do so. The process of removing the displacement baseline waveform from cumulative displacements can be simply performed using FIR filtering that removes low-frequency components or autocorrelation processing that can extract only periodic components.

In this case, a displacement baseline is removed, and a three-dimensional image having a shape different from an actual shape is created. However, since erroneous periodic components generated by manual pressing operation can be removed, the appearances of an image can be improved. A tester can be prevented from making an erroneous determination by viewing a low-accuracy image.

In this case, if the displacement baseline results from a change in the degree of pressing caused by unintentional hand movement of a tester, accurate correction processing may not be successfully performed. However, such a displacement baseline generally has a low frequency of variations and is a phenomenon which may occur at the time of general three-dimensional image acquisition without manual pressing operation for elasticity display. Accordingly, correction processing is processing effective in that if the correction processing is performed after a tester is notified to that effect, the number of times of reacquisition of data can be reduced and that the test efficiency can be improved.

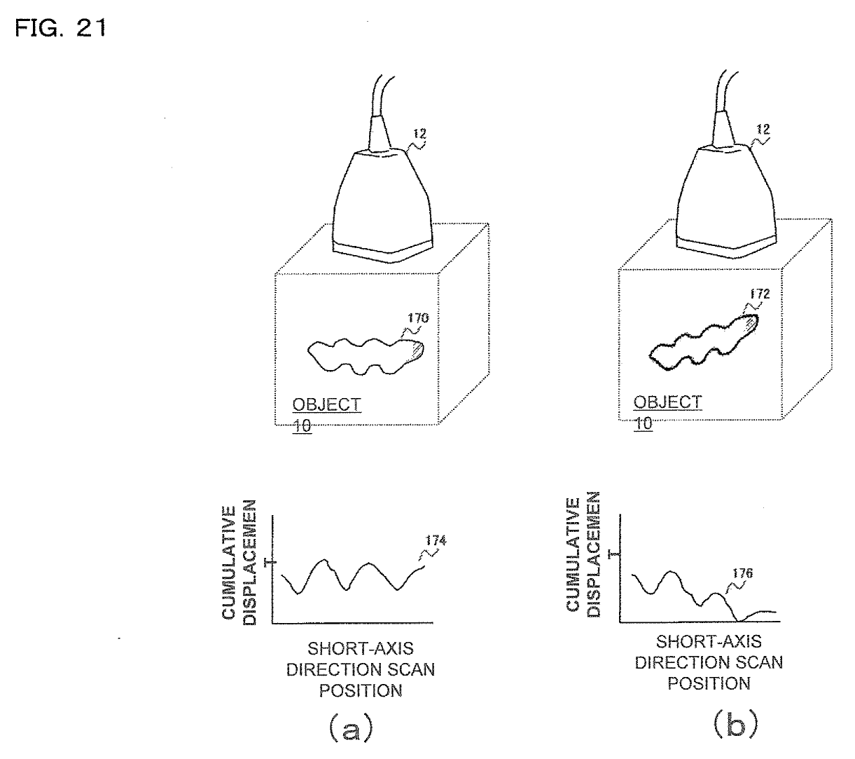

FIG. 21 show schematic figures of a three-dimensional image and a cumulative displacement graph (a) when there is no unintentional hand movement of a tester and a three-dimensional image and a cumulative displacement graph (b) when there is unintentional hand movement of a tester. A three-dimensional image 170 is a three-dimensional image including a cyclic change caused by pressing and has vertical fluctuations. In contrast, a three-dimensional image 172 is a three-dimensional image including a displacement baseline component resulting from unintentional hand movement of a tester. It can be seen that the three-dimensional image 172 not only has vertical fluctuations but also is an upward-sloping image.

Cumulative displacement graphs 174 and 176 are graphs of cumulative displacements obtained in the cases (a) and (b). The cumulative displacement graph 174 includes a cyclic change caused by pressing and has vertical fluctuations. The cumulative displacement graph 176 includes a displacement baseline component. The cumulative displacement graph 176 not only has vertical fluctuations but is a downward-sloping graph. This case is a case where a negative displacement, i.e., an upward displacement is larger than a downward displacement and may be a case where a tester gradually decreases the degree of pressing.

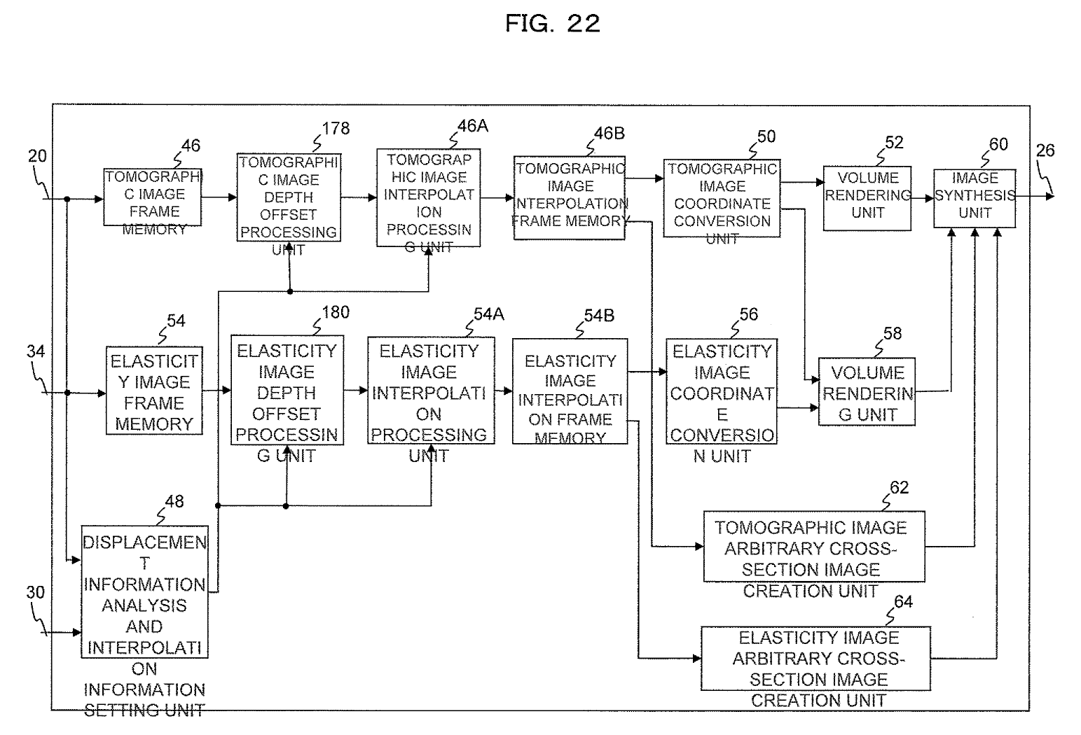

FIG. 22 shows a three-dimensional image construction unit 24 according to the present embodiment. The present embodiment is different from the first and second embodiments in that, a tomographic image interpolation processing unit 46A and an elasticity image interpolation processing unit 54A perform offset processing in a displacement direction via a tomographic image displacement offset processing unit 178 and an elasticity image displacement offset processing unit 180 when creating interpolation frames by performing interpolation processing on a tomographic image from a tomographic image frame memory 46 and an elasticity image from an elasticity image frame memory 54 according to displacement indices and short-axis position indices set by the displacement information analysis and interpolation information setting unit 48.

As described above, the displacement information analysis and interpolation information setting unit 48 estimates the waveform of a change in cumulative displacement with respect to the short axis direction, the waveform of a change in relative displacement with respect to the short axis direction, and a displacement baseline waveform. If a variation in displacement baseline is large, the displacement information analysis and interpolation information setting unit 48 displays a warning on an image display 26. The displacement information analysis and interpolation information setting unit 48 calculates a shift amount as a displacement baseline offset value for displacement baseline shifting from a variation in the displacement baseline from a pressing start frame obtained from the displacement baseline waveform, if a tester desires to do so.

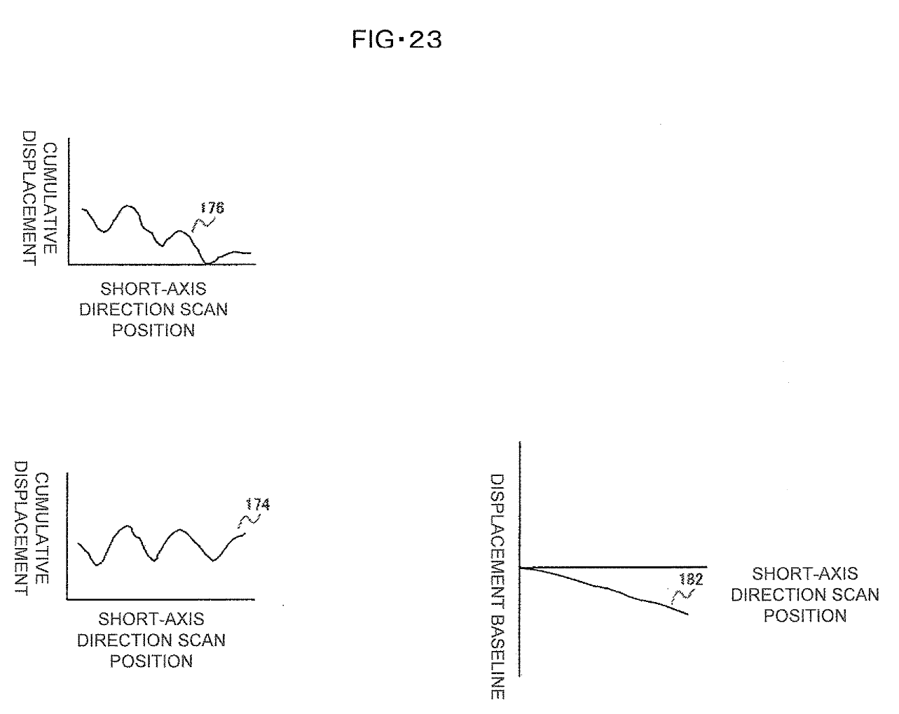

FIG. 23 shows a schematic figure of cumulative displacement graphs. When the cumulative displacement graph 176 is input, a displacement baseline graph 182 is a displacement baseline component estimated from the cumulative displacement graph 176, and the cumulative displacement graph 174 is obtained by subtracting the displacement baseline graph 182 from the cumulative displacement graph 176. The displacement information analysis and interpolation information setting unit 48 detects cumulative negative displacements from the displacement baseline graph 182, converts the negative displacements to shift amounts for respective samples, and output the shift amounts to the tomographic image displacement offset processing unit 178 and elasticity image displacement offset processing unit 180.

In the present embodiment, displacement baseline correction is performed as displacement baseline shifting in the tomographic image displacement offset processing unit 178 and elasticity image displacement offset processing unit 180. Accordingly, a cumulative displacement indicated by the cumulative displacement graph 174 shown in FIG. 23 that is obtained by subtracting a variation in the displacement baseline waveform from the waveform of a change in cumulative displacement with respect to the short axis direction for interpolation processing performed in the tomographic image interpolation processing unit 46A and elasticity image interpolation processing unit 54A.

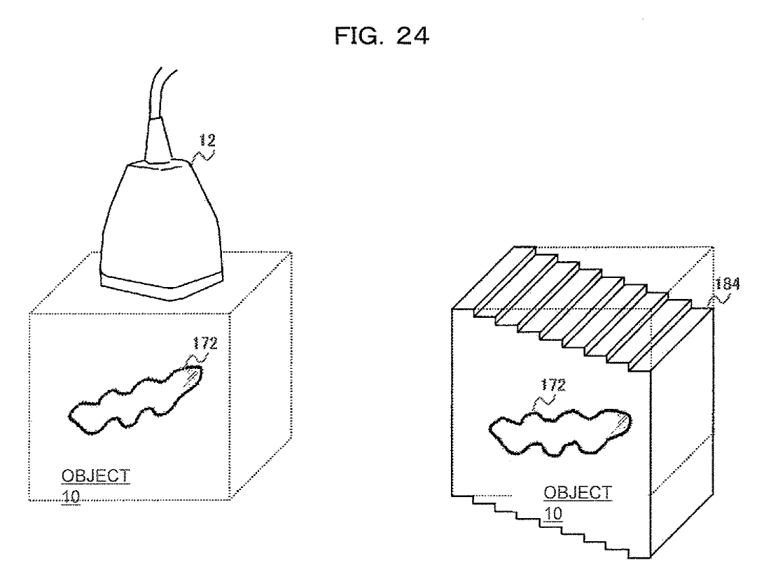

The tomographic image displacement offset processing unit 178 performs, on a piece of tomographic image data from the tomographic image frame memory 46, displacement baseline shifting that vertically shifts the piece of tomographic image data, as shown in a conceptual view 184 of displacement baseline shifting in FIG. 24, while referring to displacement baseline offset values set by the displacement information analysis and interpolation information setting unit 48 and outputs a result of the displacement baseline shifting to the tomographic image interpolation processing unit 46A. With the displacement baseline shifting, a displacement baseline component included in the three-dimensional image 172 can be removed, as shown in the three-dimensional image 170 in FIG. 21.

The elasticity image displacement offset processing unit 180 performs, on a piece of elasticity image data from the elasticity image frame memory 54, displacement baseline shifting that vertically shifts the piece of elasticity image data while referring to the displacement baseline offset values set by the displacement information analysis and interpolation information setting unit 48 and outputs a result of the displacement baseline shifting to the elasticity image interpolation processing unit 54A. By performing interpolation processing using pieces of tomographic image data and elasticity image data vertically shifted by the tomographic image displacement offset processing unit 178 and elasticity image displacement offset processing unit 180, like the first embodiment, a displacement baseline can be corrected, and a high-accuracy three-dimensional image can be created.

As has been described above, according to the present embodiment, a displacement caused when a tester gradually decreases or gradually increases pressing force due to unintentional hand movement or the like at the time of measurement can be estimated as the displacement baseline graph 182. The tester can reduce a displacement caused by unintentional hand movement or the like by changing the manner of measurement so as to clear the displacement baseline graph 182 when the displacement baseline graph 182 is displayed on a cumulative displacement graph.

Since the three-dimensional image construction unit 24 is adapted to display a warning on the image display unit if a displacement of the displacement baseline graph 182 exceeds the set value, a tester can correct pressing operation on the basis of the warning. Also, the three-dimensional image construction unit can construct a three-dimensional elasticity image or a three-dimensional tomographic image on the basis of a cumulative displacement graph from which the displacement baseline graph 182 has been removed, a three-dimensional image can be constructed by removing effects of unintentional hand movement of the tester and the like from cumulative displacements.

As a modification of the present embodiment, the present embodiment may be adapted such that only one interpolation frame is created during one cycle of a cumulative displacement. For example, in FIG. 5, the interpolation frames 88, 91, and 94 to 98 are generated. An interpolation frame can also be generated using average displacements with the same sign, i.e., one of average displacements at the time of pushing and average displacements at the time of pulling, in addition to cumulative displacements.

In this case, if interpolation frames are created using average displacements at the time of pulling, only the interpolation frames 88, 94, 96, and 98 are created. For simplification of processing, filtering may be performed on the waveform of a change with time in average displacement output from a displacement measurement unit 30, the waveform of a change with time in relative displacement may be calculated, and only one interpolation frame may be created in one pressing cycle. The modification can also be applied to the first and second embodiments.

### Fourth Embodiment

A fourth embodiment now will be described. In the first to third embodiments, a pressing displacement used for interpolation frame creation is obtained by accumulating displacements in a depth direction. A feature of the present embodiment lies in that a pressing strain is obtained by accumulating strains in the depth direction and that interpolation processing is controlled by setting a threshold value for an obtained strain. A three-dimensional image construction unit 24 obtains cumulative strains by accumulating strains in elasticity images constructed by an elasticity image construction unit 34 to generate generates volume data of the elasticity images and constructs a three-dimensional elasticity image on the basis of the generated volume data. The three-dimensional image construction unit 24 can also select ones having cumulative strains within a set range from among the plurality of elasticity images and generate volume data of the selected elasticity images. In the present embodiment, the value of a cumulative strain is expressed in percentage. Frames equal in cumulative strain (%) are selected, and interpolation is performed.

Although not shown, a displacement information analysis and interpolation information setting unit 48 is adapted to analyze an average pressing strain (average strain) output from an elasticity information calculation unit 32 and obtain a cumulative strain obtained by converting a strain from a zero-pressure state to a numerical value in terms of a strain of an object 10. Note that the term strain here refers to a cumulative strain from the zero-pressure state of the object 10.

An elasticity image interpolation processing unit 54A performs interpolation processing according to a strain-based condition in the displacement information analysis and interpolation information setting unit 48. For example, if a threshold value for a strain is 10%, the elasticity image interpolation processing unit 54A performs frame interpolation processing on only a frame having a cumulative strain different from a target cumulative strain by 10% or more. An elasticity image interpolation frame memory 54B stores interpolation frame data created by the elasticity image interpolation processing unit 54A. An elasticity image coordinate conversion unit 56 performs coordinate conversion on an output from the elasticity image interpolation frame memory 54B and creates three-dimensional volume data. A volume rendering unit 58 performs volume rendering of data having undergone coordinate conversion in the elasticity image coordinate conversion unit 56.

Similarly, a tomographic image interpolation processing unit 46A performs interpolation processing according to a strain-based condition in the displacement information analysis and interpolation information setting unit 48. A tomographic image interpolation frame memory 46B stores interpolation frame data created by the tomographic image interpolation processing unit 46A. A tomographic image coordinate conversion unit 50 performs coordinate conversion on an output from the tomographic image interpolation frame memory 46B and creates three-dimensional volume data. A volume rendering unit 52 performs volume rendering of data having undergone coordinate conversion in the tomographic image coordinate conversion unit 50.

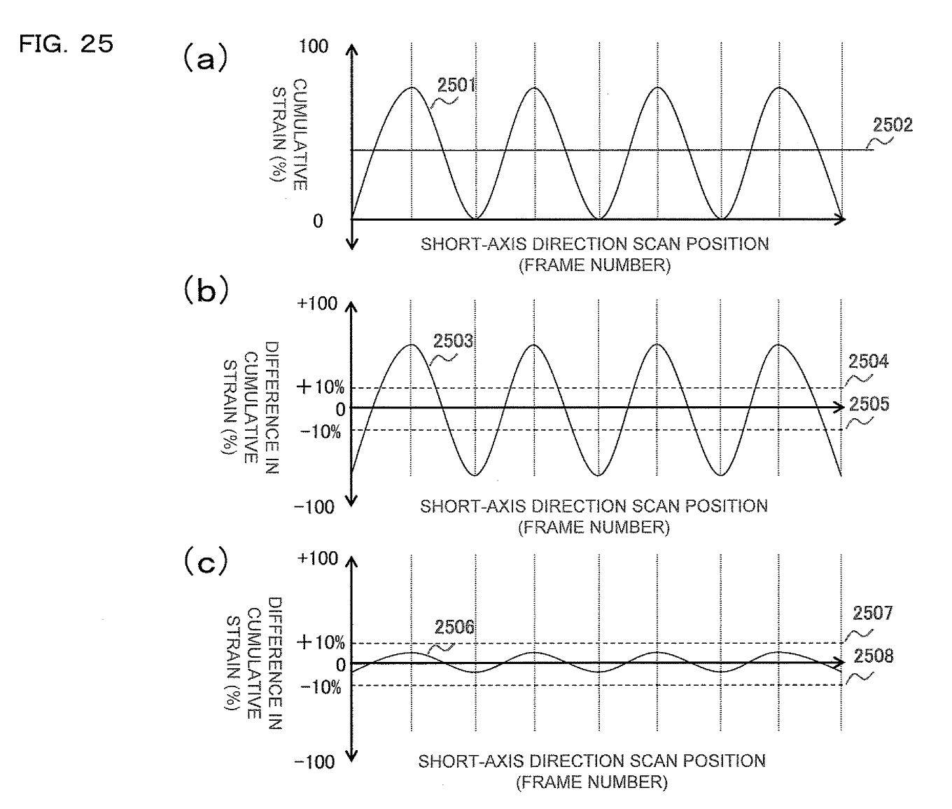

FIG. 25 show a specific example of the threshold value for a strain. A cumulative strain graph 2501 shown in FIG. 25(a) is obtained by accumulating strains obtained from displacements in a direction of time.

A cumulative strain graph 2503 shown in FIG. 25(b) is obtained by subtracting a strain at a reference line 2502 from the cumulative strain graph 2501. If 10% is specified as the threshold value, the elasticity image interpolation processing unit 54A performs interpolation processing on only a frame whose absolute value of a difference from the threshold value is not less than 10%, i.e., a frame having a strain larger than a broken line 2504 and a frame having a strain smaller than a broken line 2505. That is, since the elasticity image interpolation processing unit 54A does not perform interpolation processing on a frame whose absolute value of a difference is not more than 10%, the resolution can be maintained.

Assume that the absolute value of a difference in strain is not more than 10%, as shown in FIG. 25(c). In this case, if artifacts are too small to visually check, it is also possible to selectively correct parts with the artifacts while maintaining the resolution. In the example shown in FIG. 25(c), a graph 2506 indicates a case where the difference between a cumulative strain caused by pressing and the target cumulative strain is always not more than 10%. The elasticity image interpolation processing unit 54A performs correction processing on no region if the threshold value is 10%.

An operator can interactively operate a control panel while viewing an image, by including an adjustment dial or an adjustment button for setting the threshold value in the control panel. This allows creation of an optimum image.

The present embodiment has been described in the context of a strain obtained by normalizing a displacement by the magnitude before pressing. However, the same processing, of course, can also be performed using a displacement.

### REFERENCE SIGNS LIST

12 ultrasonic probe, 14 transmission unit, 16 reception unit, 18 phasing addition unit, 20 tomographic image construction unit, 24 three-dimensional image construction unit, 26 image display, 30 displacement measurement unit, 32 elasticity information calculation unit, 34 elasticity image construction unit, 46 tomographic image frame memory, 54 elasticity image frame memory, 48 displacement information analysis and interpolation information setting unit, 46A tomographic image interpolation processing unit, 54A elasticity image interpolation processing unit, 60 image synthesis unit, 84 cumulative displacement graph, 182 displacement baseline

1. An ultrasonic diagnostic apparatus, comprising:
an ultrasonic probe configured to transmit/receive ultrasonic waves to/from an object in contact with the object;
a transmission/reception unit configured to perform reception processing on a reflection echo signal from the object and measures RF signal frame data;
a displacement measurement unit configured to obtain a displacement on the basis of the RF signal frame data measured by the transmission/reception unit;
an elasticity image construction unit configured to construct an elasticity image on the basis of the displacement obtained by the displacement measurement unit; and
a three-dimensional image construction unit configured to generate elasticity image volume data by obtaining a cumulative displacement or a cumulative strain by accumulating a displacement or a strain in the elasticity image constructed by the elasticity image construction unit, and constructs a three-dimensional elasticity image on the basis of the generated volume data.

2. The ultrasonic diagnostic apparatus according to claim 1, wherein the three-dimensional image construction unit obtains a cumulative displacement or a cumulative strain by accumulating a displacement or a strain of a living organism tissue in the elasticity image sequentially constructed by the elasticity image construction unit, selects one having the cumulative displacement or the cumulative strain within a set range from among a plurality of the elasticity images, and generates volume data of the selected elasticity image.

3. The ultrasonic diagnostic apparatus according to claim 2, wherein the three-dimensional image construction unit generates and interpolates an elasticity image corresponding to a desired cumulative displacement on the basis of elasticity images located next in short-axis scan position to the elasticity image corresponding to the desired cumulative displacement and a relationship between cumulative displacements and the short-axis scan positions of the elasticity images and generates volume data including the interpolated elasticity image.

4. The ultrasonic diagnostic apparatus according to claim 2, wherein if an elasticity image corresponding to a desired cumulative displacement is not obtained in one pressing cycle, the three-dimensional image construction unit generates and interpolates the elasticity image corresponding to the desired cumulative displacement on the basis of elasticity images obtained in respective pressing cycles immediately preceding and following the one pressing cycle and a relationship between cumulative displacements and short-axis scan positions of the elasticity images and generates volume data including the interpolated elasticity image.

5. The ultrasonic diagnostic apparatus according to claim 2, wherein the three-dimensional image construction unit creates a cumulative displacement graph indicating a relationship between the cumulative displacement and a position in a short-axis scan direction of an elasticity image at the cumulative displacement.

6. The ultrasonic diagnostic apparatus according to claim 1, comprising
a tomographic image construction unit configured to sequentially construct tomographic images of a living organism tissue on the basis of a plurality of pieces of RF signal frame data measured by the transmission/reception unit, wherein
the three-dimensional image construction unit associates the plurality of tomographic images output from the tomographic image construction unit with a respective number of the displacements output from the displacement measurement unit, obtains respective cumulative displacements for the tomographic images by accumulating the associated displacements, selects one having the cumulative displacement within a set range from among the plurality of tomographic images to generate volume data of the selected tomographic image, and constructs a three-dimensional tomographic image on the basis of the generated volume data.

7. The ultrasonic diagnostic apparatus according to claim 6, wherein the three-dimensional image construction unit generates and interpolates a tomographic image corresponding to a desired cumulative displacement on the basis of tomographic images located next in short-axis scan position to the tomographic image corresponding to the desired cumulative displacement and a relationship between cumulative displacements and the short-axis scan positions of the tomographic images and generates volume data including the interpolated tomographic image.

8. The ultrasonic diagnostic apparatus according to claim 6, wherein if a tomographic image corresponding to a desired cumulative displacement is not obtained in one pressing cycle, the three-dimensional image construction unit generates and interpolates the tomographic image corresponding to the desired cumulative displacement on the basis of tomographic images obtained in respective pressing cycles immediately preceding and following the one pressing cycle and a relationship between cumulative displacements and short-axis scan positions of the tomographic images and generates volume data including the interpolated tomographic image.

9. The ultrasonic diagnostic apparatus according to claim 6, wherein the three-dimensional image construction unit obtains an elasticity value on the basis of the three-dimensional elasticity image, converts the three-dimensional elasticity image to an image display format corresponding to the elasticity value, and superimposes the three-dimensional elasticity image on the three-dimensional tomographic image.

10. The ultrasonic diagnostic apparatus according to claim 5, wherein the three-dimensional image construction unit includes an image display unit configured to display a screen including the cumulative displacement graph and at least one of an elasticity image constructed by the elasticity image construction unit and a tomographic image constructed by the tomographic image construction unit, and a three-dimensional elasticity image and a three-dimensional tomographic image constructed by the three-dimensional image construction unit.

11. The ultrasonic diagnostic apparatus according to claim 6, wherein the three-dimensional image construction unit displays a screen including an elasticity image or a tomographic image at an arbitrary cross-section of the three-dimensional elasticity image or the three-dimensional tomographic image and a composite image of the elasticity image and the tomographic image.

12. The ultrasonic diagnostic apparatus according to claim 5, wherein
when change of pressing force applied to the object and movement of a cross-sectional position in the short-axis scan direction are manually performed while the ultrasonic probe is grasped,
the three-dimensional image construction unit extracts a displacement caused by imbalance in the pressing force on the basis of the cumulative displacement graph to create a displacement baseline and displays the displacement baseline on the cumulative displacement graph displayed on the image display unit.

13. The ultrasonic diagnostic apparatus according to claim 12, wherein the three-dimensional image construction unit displays a warning on the image display unit when a displacement of the displacement baseline exceeds a set value.

14. The ultrasonic diagnostic apparatus according to claim 12, wherein the three-dimensional image construction unit constructs the three-dimensional elasticity image or the three-dimensional tomographic image on the basis of a cumulative displacement graph obtained by removing the displacement baseline from the cumulative displacement graph.

15. An image construction method, comprising:
performing reception processing on a reflection echo signal from an object via an ultrasonic probe configured to transmit/receive ultrasonic waves to/from the object while being in contact with the object and measuring RF signal frame data;
obtaining a displacement on the basis of the measured RF signal frame data;
constructing an elasticity image on the basis of the obtained displacement; and
generating elasticity image volume data by obtaining a cumulative displacement by accumulating a displacement in the constructed elasticity image, and constructing a three-dimensional elasticity image on the basis of the generated volume data.
